(19) United States
(12) Patent Application Publication
(10) Pub. No.: US 2026/0365725 A1
(43) Pub. Date: Nov. 19, 2026

(54) MONOCLONAL ANTIBODIES AGAINST NEONATAL NAV1.5

(71) Applicant: Celex Oncology Innovations Limited, London (GB)

(72) Inventors: Mustafa Bilgin Ali Djamgoz, London (GB); Carsten Faltum, London (GB)

(73) Assignee: Celex Oncology Innovations Limited, London (GB)

(21) Appl. No.: 19/647,332

(22) Filed: Apr. 14, 2026

Related U.S. Application Data
(63) Continuation of application No. 17/794,083, filed on Jan. 21, 2021, now Pat. No. 12,624,101.

(30) Foreign Application Priority Data
Jan. 21, 2020 (EP) 20152789.2

(51) Int. Cl. C07K 16/28; A61P 35/00; C12N 15/63

(52) U.S. Cl. CPC C07K 16/28; A61P 35/00; C12N 15/63; C07K 2317/24; C07K 2317/565

(57) ABSTRACT

Describe herein are binding molecules which specifically bind to neonatal 5-exon splice variants of the α subunit of Naᵥ1.5 (nNaᵥ1.5), including monoclonal antibodies and antigen-binding fragments, bispecific antibodies and antibody-drug conjugates derived from such monoclonal antibodies, and NK and T cells comprising chimeric antigen receptors derived from such monoclonal antibodies, and their use in diagnostic and therapeutic methods.

### RELATED APPLICATIONS

This application is a continuation of U.S. application Ser. No. 17/794,083, filed Jul. 20, 2022, which is a national stage filing under 35 U.S.C. 371 of PCT/EP2021/051317, filed Jan. 21, 2021, which was published by the International Bureau in English on Jul. 29, 2021, and which claims priority from European Application No. 20152789.2, filed Jan. 21, 2020, each of which is hereby incorporated in its entirety by reference in this application.

### REFERENCE TO A SEQUENCE LISTING SUBMITTED ELECTRONICALLY AS AN XML FILE

The instant application contains a Sequence Listing which has been submitted in xml format via USPTO Patent Center and is hereby incorporated by reference in its entirety. Said xml copy, created on Apr. 14, 2026, is named I88449_1500US.C1_00109_Seq_List.xml, and is 25000 bytes in size.

### FIELD OF THE INVENTION

The present invention relates to the field of cancer therapy. In particular, the present invention relates to the field of treatment of metastasizing cancers and to therapeutic means and methods.

### BACKGROUND OF THE INVENTION

Several major human carcinomas express functional voltage-gated Na⁺ channels (VGSCs) which promote their cellular invasiveness in vitro and metastasis in vivo (Djamgoz et al., 2019). In humans, there are nine different VGSC alpha subunits or “Nav” proteins (Naᵥ1.1 to Naᵥ1.9), encoded by the genes SCN1A, SCN2A, SCN3A, SCN4A, SCN5A, SCN8A, SCN9A, SCN10A and SCN11A, respectively. The alpha subunit of VGSCs is a transmembrane protein with 4 repetitive transmembrane domains (termed DI, DII, DIII and DIV), which each contains 6 transmembrane spanning sections that are termed S1-S6. Na⁺ ions flow through a pore formed by S5 and S6 combined from all 4 domains. The S4 segment plays a central role in voltage sensing and channel activation. The smaller beta subunits contain an N-terminal extracellular immunoglobulin (Ig) loop, a transmembrane domain and an intracellular domain, and function as regulatory and adhesion molecules.

Human Naᵥ1.5 is encoded by the gene SCN5A, a highly conserved gene located on human chromosome 3, where it spans more than 100 kb. The gene consists of 28 exons, of which exon 1 and in part exon 2 form the 5′ untranslated region (5′UTR), and exon 28 forms the 3′ untranslated region (3′UTR) of the RNA. More than 10 different splice isoforms have been described for SCN5A, of which several harbour different functional properties. Importantly, Naᵥ1.5 is developmentally regulated via alternative splicing of exon 6, giving rise to ‘adult’ and ‘neonatal’ or ‘foetal’ variants of the Nav1.5 protein that differ in the S3-S4 region of DI by several amino acids. The foetal and adult form of the protein can have 7 amino acid differences in the DI:S3-S4 region of the channel protein.

It has previously been found that the foetal/neonatal isoform of Naᵥ1.5 (nNaᵥ1.5) is associated with metastatic cancers and in vitro data have demonstrated that it is possible to interfere with the metastatic ability of malignant cells by blocking nNaᵥ1.5 (Djamgoz et al., 2019).

Chioni et al. (2005) describes the generation of an anti-peptide polyclonal antibody, named NESOpAb, which specifically recognised ‘neonatal’ but not ‘adult’ Naᵥ1.5 when tested on cells specifically over-expressing one or other of these Naᵥ1.5 spliced forms.

However, there is still a need for therapeutic and diagnostic means and methods for cancers, particularly cancers associated with nNav1.5 expression.

It is an object of embodiments of the invention to provide such therapeutic and diagnostic means and methods.

### SUMMARY OF THE INVENTION

The present inventor has identified monoclonal antibodies which specifically bind to nNaᵥ1.5.

So, in one aspect, the present invention provides a monoclonal antibody, or an antigen-binding fragment thereof, which specifically binds to neonatal 5′-exon splice variants of the α subunit of Nav1.5 (nNaᵥ1.5), optionally comprising human constant heavy (CH) and constant light (CL) domains and/or human framework (FR) regions.

In one aspect, the present invention provides a chimeric antigen receptor (CAR) which specifically binds to neonatal 5′-exon splice variants of the α subunit of Nav1.5 (nNaᵥ1.5), optionally comprising an antigen-binding fragment derived from the monoclonal antibody of the preceding aspect.

These and other aspects and embodiments of the invention are disclosed in more detail below.

### LEGENDS TO THE FIGURES

FIG. 1 shows Nav1.5 splice variant sequences encoded by exon 6 of the SCN5A gene and a consensus sequence (SEQ ID NO:4).

FIG. 2 shows the results of immunocytochemical tests, using mAbs obtained from hybridomas and EBNA cell lines stably expressing nNav1.5 or aNav1.5. See Example 1 for details.

FIG. 3 shows the results of patch-clamp recordings performed on EBNA cells stably expressing neonatal Nav1.5 as previously described by Chioni et al. (2005). The blocking ability of mAb candidates (supernatants) was tested by short-term application using a ‘puff’ pipette whilst clamping the cells at different holding potentials. The figure shows the dose dependent blockage of nNav1.5 currents by the A3 and C2 mAbs. Each data point denotes means+/−standard error (n=4-6). The dotted horizontal line denotes ‘null effect’ for ease of comparison.

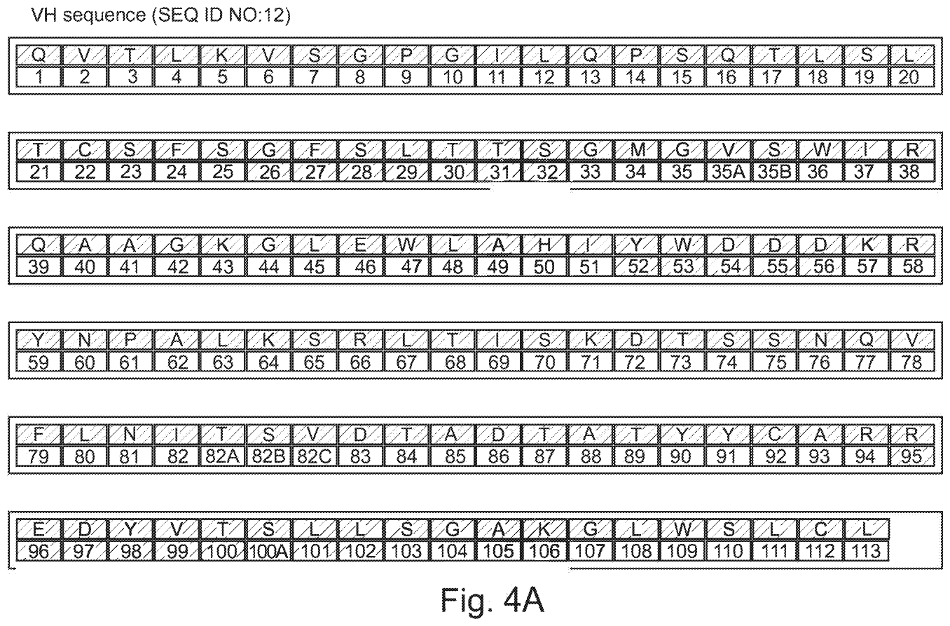

FIG. 4A and FIG. 4B shows the VH (FIG. 4A) and VL (FIG. 4B) amino acid sequences of the A3 antibody (SEQ ID NO:12 and 13, respectively), with the amino acid positions numbered and the CDR residues (shaded amino acid positions) defined according to the Kabat numbering system, as described elsewhere herein.

### DETAILED DISCLOSURE OF THE INVENTION

Cells expressing neonatal splice forms of the α chain of Naᵥ1.5 (in the following collectively termed nNaᵥ1.5) are exclusively or almost exclusively found in metastatic cancer tissues. For instance, no expression was found in healthy colon, small intestine, stomach, prostate, brain, skeletal muscle, and cardiac muscle. This finding demonstrates that therapeutic approaches that target this particular protein will target a tumour specific antigen. In turn this means that targeting of this particular antigen is highly likely to be clinically safe due to the low abundancy/absence of the antigen in normal tissue. nNaᵥ1.5 has also been found to be strongly expressed by human colon polyps, consistent with their pre-cancerous nature.

It has also been found that a polyclonal antibody (NESOpAb) blocks nNaᵥ1.5 current with two orders of magnitude higher affinity compared with the currents generated by (i) the closest related protein, adult Naᵥ1.5 (aNaᵥ1.5) and (ii) Naᵥ1.7. Further, the binding of the antibody is use dependent, i.e. blockage of nNaᵥ1.5 by NESOpAb is much more efficient when the channel is opening and closing rather than when it is just closed. Moreover, the binding of the NESOpAb antibody is voltage dependent, meaning that the binding becomes more effective as the membrane potential is depolarized. This latter characteristic renders a nNaᵥ1.5 specific antibody even more specific to cancer cells, which are well known to have depolarized resting potentials.

The binding of the antibody also leads to hyperpolarization of nNaᵥ1.5 “availability”, making channel activation less likely.

Lysine (K) at position 211 in the middle of the spliced region of nNaᵥ1.5 has been found to be critical for the binding of NESOpAb. Mutating it back to aspartate (D) as in the adult (double-charge change), reduced blocking efficacy to the level of aNav1.5.

As described in Example 1, monoclonal antibodies binding to nNav1.5 have now been identified. A monoclonal antibody according to the present invention can be defined by its binding specificity, particularly by its specific binding to neonatal 5′-exon splice variants of the α subunit of nNaᵥ1.5. Typically, the monoclonal antibody binds more readily to a nNav1.5 (e.g., a nNav1.5 comprising the amino acid sequence of residues 206 to 219 of SEQ ID NO:2) than to an adult 3′-exon splice variant of the α subunit of Nav1.5 (aNav1.5) (e.g., comprising the amino acid sequence of residues 206 to 219 of SEQ ID NO:1), such as, for example, with a factor of at least 1.5:1, at least 2:1, at least 3:1, at least 4:1, at least 5:1, at least 10:1 or at least 20:1, to the nNav1.5 as compared to the aNav1.5 (nNav1.5:aNav1.5). The monoclonal antibody may also or alternatively bind nNav1.5 with at least 2 times, such as at least 3 times, such as at least 4 times, such as at least 5 times, such as at least 10 times, such as at least 20 times, such as at least 100 times higher binding affinity compared to binding to adult 3′-exon splice variants of the α subunit of adult Nav1.5 (aNav1.5). For example, the monoclonal antibody may bind nNaᵥ1.5 comprising SEQ ID NO:2 with a Ko at most about half, such as at most about one third, such as at most about one fourth, such as at most about one fifth, such as at most about one tenth, such as at most about one twentieth, such as at most one hundredth, of that of its binding to adult 3′-exon splice variants of the α subunit of Naᵥ1.5 (aNaᵥ1.5) comprising SEQ ID NO:1.

In one embodiment, the nNav1.5 to which the monoclonal antibody binds comprises an amino acid sequence that differs from SEQ ID NO:1 in at least amino acid residue 211, such as in amino acid residues 206, 207, 209, 210, 211, 215, and 234. For example, the nNav1.5 may comprise amino acid residues V, S, N, I, K, L, and P in positions 206, 207, 209, 210, 211, 215, and 234 respectively, where aNav1.5 comprises the amino acid residues T, T, F, V, D, V, and S in the same/corresponding positions. In a specific embodiment, the nNav1.5 is human nNav1.5. In another specific embodiment, the monoclonal antibody binds to an epitope in nNav1.5, which has the residue Lys (K) in position 211. In some embodiments, the monoclonal antibody binds to a nNav1.5 comprising the peptide segment VSENIKLGNLSALR, corresponding to residues 206 to 219 of SEQ ID NO:2. In one specific embodiment, the monoclonal antibody binds to SEQ ID NO:5 (herein referred to as “NESO peptide”).

The monoclonal antibody may also be characterized by comprising human sequences, e.g., human CH and CL domains. In some embodiments, all constant domains, framework regions, or constant and framework regions of the monoclonal antibody, are human. In certain embodiments, the monoclonal antibody is a chimeric antibody, a humanized antibody or a fully human antibody. Antigen-binding fragments (Fab) of such a monoclonal antibody, including Fabr-fragments, Fab′ fragments and single-chain Fv (scFv) fragments, are also provided. A scFv typically comprises the variable portions of an immunoglobulin heavy and light chain, fused by a flexible linker.

Chimeric antigen receptors (CARs), artificial T cell receptors which are also known as chimeric T cell receptors and/or chimeric immunoreceptors, are engineered receptors, which graft a selected specificity onto an immune effector cell. These receptors may, for example, be used to graft the specificity of a monoclonal antibody onto a T cell or Natural Killer (NK) cell, using, e.g., retroviral vectors to transduce the cells with coding sequences. Such cells are under investigation as a therapy for cancer, using a technique called adoptive cell transfer. Briefly, T cells are removed from a patient and modified so that they express a CAR specific to the patient's particular cancer. Upon reintroduction into the patient, the T cells can then recognize and kill the cancer cells. The design, construction and therapeutic use of CARs have been reviewed by, e.g., Dotti et al. (2014) and Sadelain et al. (2013).

As set forth herein, the CARs of the present invention comprise a binding portion which specifically binds to neonatal 5′-exon splice variants of the α subunit of Nav1.5 (nNav1.5), a transmembrane domain and an endodomain.

The binding portion may advantageously comprise an antigen-binding portion of a monoclonal antibody, such as an antigen-binding fragment, which specifically binds to neonatal 5′-exon splice variants of the α subunit of nNav1.5. Preferably, the antigen-binding fragment is an antigen-binding fragment of a monoclonal antibody according to an aspect or embodiment described herein. Suitable formats of antigen-binding fragments include, without limitation, Fab-fragments and scFv fragments. The scFv may also include an N-terminal signal peptide to direct the nascent protein to the endoplasmic reticulum and subsequent surface expression.

The transmembrane domain is typically a hydrophobic alpha helix derived from the original molecule of the endodomain that protrudes into the cell and transmits the desired signal. In embodiments where the endodomain comprises two or more portions that are derived from different molecules as described below, the transmembrane portion typically derives from the same molecule as the most membrane-proximal portion of the endodomain.

The domains of the CAR may be fused directly to each other or may be joined by peptide linkers. For example, the binding domain, e.g., the scFv, may be fused to the transmembrane domain via a peptide linker, typically a flexible peptide allowing the binding domain to orient in different directions to enable binding to the nNav1.5 antigen. In some embodiments, the peptide linker joining a binding portion which is an antigen-binding fragment and the transmembrane domain may comprise the hinge portion of the constant region of an immunoglobulin, such as IgG1 or IgG4.

The endodomain is the signalling portion of the CAR construct, sending an activation signal to the T or NK cell upon binding of the binding portion to the antigen. Typically, the endodomain comprises a signalling domain derived from a T cell receptor molecule, preferably CD3-ζ (CD3-zeta) which contains immunoreceptor tyrosine-based activation motifs (ITAMs) transmitting an activation signal. In some embodiments, the endodomain further comprises one or more costimulatory molecules, typically at least one of CD27, CD28, 4-IBB and OX40, to enhance the activation signal.

In some embodiments, the different portions of the CAR construct are arranged, from N- to C-terminus, as follows: nNav1.5-binding domain/hinge/transmembrane domain/endodomain, wherein the nNav1.5-binding domain may be a scFv, the hinge may comprise an IgG1 or IgG4 hinge region, and the transmembrane domain and the most membrane-proximal molecule of the endodomain may derive from the same molecule. The endodomain may, for example, comprise, from membrane-proximal to membrane-distal, CD3-zeta, CD28/CD3-zeta, CD28/4-IBB/CD3-zeta.

An exemplary CAR vector, such as a retroviral vector which includes a nucleic acid encoding a CAR, can be transfected into T cells and/or NK cells. In some embodiments, the T cells are CD8+ T cells. In some embodiments, the T cells are CD4+ T cells.

### DEFINITIONS

It is to be understood that this invention is not limited to particular aspects and embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, technical and scientific terms used herein have the same meaning as commonly understood by one of ordinary skill in the art to which this invention belongs.

A “Naᵥ1.5” denotes the α unit of a voltage gated sodium channel (VGSC), where nNaᵥ1.5 is the “neonatal form” (Chioni et al., 2005; Fraser et al., 2005; UniprotKB Q14524 (SCN5A_HUMAN)). The amino acid sequence of the α subunit of Naᵥ1.5 is set forth herein as SEQ ID NO:1 (UniProt Q14524-1).

As used herein, “neonatal Nav1.5” (nNav1.5), also referred to herein as “foetal Nav1.5” or “fetal Nav1.5”, comprises an α subunit amino acid sequence that differs from SEQ ID NO:1 in at least amino acid residue 211, such as in amino acid residues 206, 207, 209, 210, 211, 215, and 234. Preferably, in nNav1.5, the amino acid at position 211 of SEQ ID NO:1 is K (Lys). For example, the nNav1.5 may comprise amino acid residues V, S, N, I, K, L, and P in positions 206, 207, 209, 210, 211, 215, and 234 respectively, where aNav1.5 comprises the amino acid residues T, T, F, V, D, V, and S in the same/corresponding positions. In one embodiment, in the neonatal variant, residues 206-211 of SEQ ID NO:1 are changed from TTEFVD→VSENIK, optionally wherein, in the neonatal variant, the amino acid residue at position 215 is changed from V→L and/or the amino acid residue at position 234 is changed from S→P. A specific example of a nNav1.5 α subunit amino acid sequence is illustrated in SEQ ID NO:2 in the Sequence Table herein (UniProtKB—H9KVD2 (H9KVD2_HUMAN)), where the bold, underlined segment corresponds to (alternatively spliced) exon 6.

As used herein, the term “binding molecule” refers in its broadest sense to a molecule that specifically binds to an antigenic determinant, such as an epitope in nNav1.5. A binding molecule typically comprises one or more “antigen-binding domains” as described herein. Non-limiting examples of binding molecules include an antibody, a fragment thereof, an antibody-drug conjugate thereof, a bispecific or other multispecific antibody thereof, and a CAR comprising an antigen-binding portion of an antibody, which retain the antigen-specific binding of the antibody.

An “antibody” or “immunoglobulin”, used interchangeably herein, includes at least a variable domain of a heavy chain and a variable domain of a light chain, except in the case of a camelid antibody which may only include a variable domain of a heavy chain. The structure of immunoglobulins has been well characterized. See for instance Fundamental Immunology Ch. 7 (Paul, W., ed., 2nd ed. Raven Press, N.Y. (1989)). Unless contradicted by context, the term “antibody” as used herein encompasses any molecule ranging from a small antigen-binding fragment of an antibody to a full-sized antibody, typically including two complete heavy chains and two complete light chains. Heavy chains are classified as gamma, mu, alpha, delta, or epsilon, (γ, μ, α, δ, ε), which in turn determines the “isotype” of the antibody as IgG, IgM, IgA IgG, or IgE, respectively. There are also subclasses among each isotype (e.g., γ1-γ4) or α1-α2). Modified versions of each of these immunoglobulins and their respective subclasses, including both naturally occurring allotypes as well as mutants or fragments modulating properties of interest, are also well known in the art.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells.

Each heavy chain is typically comprised of a heavy chain variable (VH) region and a heavy chain constant (CH) region. The CH region typically is comprised of three domains, CH1, CH2, and CH3. The heavy chains are typically inter-connected via disulfide bonds in the so-called “hinge region”. Each light chain is typically comprised of a light chain variable (VL) region and a light chain constant region, the latter typically comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL region is typically composed of three CDRs and four FRs, arranged from amino-terminus to carboxy-terminus in the following order: FR1, CDR1, FR2, CDR2, FR3, CDR3, FR4. The FRs act to form a scaffold that provides for positioning the CDRs in correct orientation by inter-chain, non-covalent interactions, whereas the binding domain formed by the positioned CDRs defines a surface complementary to the epitope on the immunoreactive antigen. This complementary surface promotes the non-covalent binding of the antibody to its cognate epitope.

The amino acids that make up the CDRs and the FRs, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties). Application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody. Unless use of the Kabat numbering system is explicitly noted, however, consecutive numbering is used for all amino acid sequences in this disclosure.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al.

Various forms of antigen-binding fragments of antibodies are well-known in the art, and include, e.g., Fab, Fab′ and F(ab′)₂, Fd, Fvs, single-chain Fvs (scFv), single-chain antibodies and disulfide-linked Fvs (sdFv). ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen binding domain more readily than it would bind to a random, unrelated epitope. As used herein, an antibody or antigen-binding fragment thereof which “specifically binds to neonatal 5′-exon splice variants of the α subunit of Nav1.5 (nNav1.5)” typically means that the antibody or antigen-binding fragment binds more readily to a nNav1.5 than it does to aNav1.5. In some embodiments, the nNav1.5 comprises the peptide segment VSENIKLGNLSALR, corresponding to residues 206 to 219 of SEQ ID NO:2; the peptide segment of SEQ ID NO:3, corresponding to exon 6. In some embodiments, the nNav1.5 comprises SEQ ID NO:2 or at least the extracellular portion of SEQ ID NO:2 that comprises residues 206 to 219. An aNav1.5 may comprise SEQ ID NO:1 or at least the extracellular portion of SEQ ID NO:1 that comprises residues 206 to 219.

An antibody or antigen-binding fragment thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope by at least 90%, at least 80%, at least 70%, at least 60%, or at least 50%.

As used herein, the term “affinity” or “binding affinity” refers to a measure of the strength of the binding of an individual epitope with one or more binding domains, e.g., of an immunoglobulin molecule. See, e.g., Harlow et al., Antibodies: A Laboratory Manual, (Cold Spring Harbor Laboratory Press, 2nd ed. 1988) at pages 27-28. The affinity of an antibody or fragment can often be described or specified in terms of their dissociation constant or Ko in binding to the antigen. The Ko of an antibody specifically binding to an antigen can be, for example, no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, or 5×10⁻⁹ M, and can also be for, example, no greater than 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

As used herein, the term “chimeric antibody” usually refers to an antibody in which at least part of the heavy chain constant region of a non-human antibody is replaced by the corresponding heavy chain constant region of a human antibody of a specific isotype.

A “humanized” antibody is an engineered antibody in which one or more “donor” CDRs from a non-human antibody of known specificity are grafted into a human heavy or light chain framework region is referred to herein as a “humanized antibody.” In certain aspects not all of the CDRs are replaced with the complete CDRs from the donor variable region and yet the antigen binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein, unless contradicted by context, the terms “monoclonal antibody”, “mAb”, “monoclonal antibody composition” or the like refer to a preparation of antibody molecules of a single molecular composition. A monoclonal antibody preparation can be generated by a hybridoma, e.g., a B cell obtained from an animal fused to an immortalized cell, or by means of recombinant expression of nucleic acid(s) encoding the antibody in a host cell.

A monoclonal antibody or antigen-binding fragment thereof can be monovalent, bivalent or multivalent, referring to the number of antigen-binding domains in the given molecule (i.e., one, two and more than one, respectively). An antibody in standard IgG format, for example, is bivalent. A bivalent or multivalent binding molecule can be monospecific, i.e., where all of the antigen-binding domains bind to the same epitopes and are typically the same, or can be bispecific or multispecific, e.g., where two or more binding domains bind to different epitopes on the same antigen, or bind to entirely different antigens, because of different antigen-binding domains.

As used herein, the terms “fragment,” “variant,” “derivative” and “analog” of a reference compound, e.g., a polypeptide, refer to modified versions of that reference compound which retain at least some of the properties of the reference compound. For example, a “fragment” or a “variant” of a reference native antibody or polypeptide which, for example, provides for specific binding to an antigen, typically retains that ability. Variants of, e.g., a polypeptide include fragments as described above, and also polypeptides with altered amino acid sequences due to amino acid substitutions, deletions, or insertions. Variant polypeptides can comprise conservative or non-conservative amino acid substitutions, deletions or additions.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

As used herein, a sequence that is “similar” to a reference sequence typically has a sequence identity of at least about 80%, such as at least about 85%, such as at least about 90%, such as at least about 95%, such as at least about 96%, 97%, 98% or 99% to the reference sequence; whereas a sequence “identical” to a reference sequence has a sequence identify of 100% to the reference sequence or at least a segment thereof.

The percent identity between two sequences is a function of the number of identical positions shared by the sequences (i.e., % homology=#of identical positions/total #of positions×100), taking into account the number of gaps, and the length of each gap, necessary for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444 453 (1970) algorithm.

### SPECIFIC EMBODIMENTS OF THE INVENTION

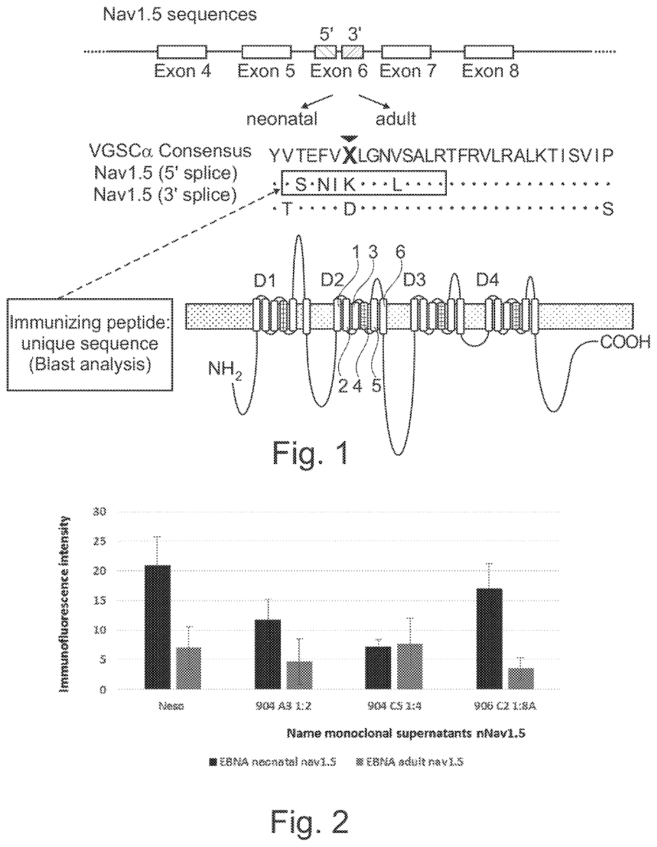

As described in Example 1, monoclonal antibodies were raised against the peptide described in Chioni et al., (2005); NH₂-VSENIKLGNLSALRC-amide (SEQ ID NO:5), herein referred to as “NESO peptide” (Chioni et al., 2005) and were found to specifically bind to nNav1.5 (FIG. 1).

The present invention relates to a monoclonal antibody which specifically binds to neonatal 5′-exon splice variants of the α subunit of Naᵥ1.5 (nNaᵥ1.5), to antigen-binding fragments and CARs derived from such monoclonal antibodies as well as to their use in diagnostic and therapeutic methods. These are set out in more detail below, wherein the term “binding molecule of the invention” can refer to any one or more of a monoclonal antibody, antigen-binding fragment thereof, or a CAR (optionally in the form of a T or NK cell comprising the CAR), which specifically binds to nNaᵥ1.5, as described in any aspect or embodiment herein.

### Binding Molecules

A binding molecule according any aspect or embodiment comprises a binding domain allowing for specific binding to nNav1.5. Typically, a binding molecule comprises one or more antibody variable domains, such as a VH region and a VL region, comprising VH CDR 1-3 and VL CDR 1-3 forming the binding domain.

In some embodiments, the binding molecule is a monoclonal antibody which specifically binds to nNaᵥ1.5.

In some embodiments, the binding molecule is an antigen-binding fragment of a monoclonal antibody, which specifically binds to nNaᵥ1.5.

In some embodiments, the binding molecule is a CAR (optionally in the form of a T or NK cell comprising the CAR), which specifically binds to nNaᵥ1.5.

In some embodiments, the binding molecule specifically binds to a nNav1.5 comprising the peptide VSENIKLGNLSALR, corresponding to residues 206 to 219 of SEQ ID NO:2.

In some embodiments, the binding molecule specifically binds to a Nav1.5 comprising YVSENIKLGNLSALRTFRVLRALKTISVIP (SEQ ID NO:3), which is the amino acid sequence corresponding to exon 6 of a nNav1.5.

In some embodiments, the binding molecule specifically binds the NESO peptide (SEQ ID NO:5), described in Chioni et al. (2005).

In some embodiments, the binding molecule specifically binds to a nNaᵥ1.5 comprising at least the extracellular portion of SEQ ID NO:2.

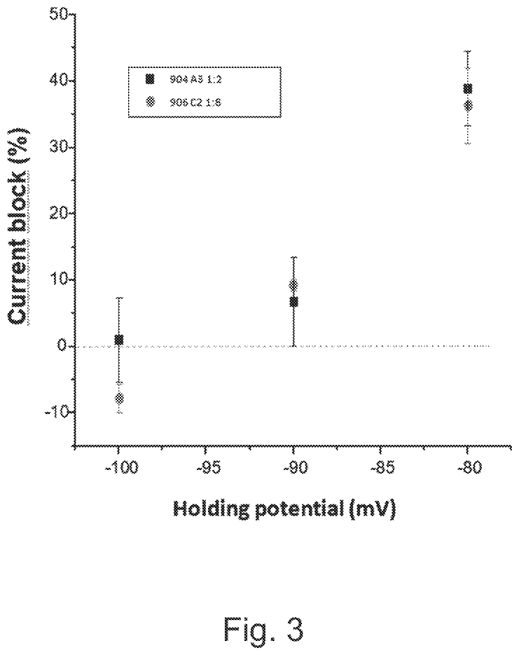

In some embodiments, the binding molecule specifically binds to a cell expressing a nNav1.5 as defined herein, e.g., a Nav1.5 comprising SEQ ID NO:2. The preparation of a nNav1.5 expressing cell line as well as an aNav1.5-expressing control cell line, both prepared by transfecting EBNA-293 cells with DNA encoding nNav1.5 and aNav1.5, respectively, is described in Chioni et al. (2005). These are suitable for evaluating specific binding to nNav1.5 according to standard methods known in the art. See also Example 1 and FIG. 2.

In some embodiments, the binding molecule binds more readily to a first cell or cell preparation expressing a nNav1.5 than to a second cell or cell preparation expressing an aNav1.5, e.g., wherein the cell or cell preparation derives from EBNA-293 cells respectively transfected with DNA encoding a nNav1.5 and an aNav1.5 as described in Chioni et al. (2005) and is incubated with the binding molecule under appropriate conditions. The binding molecule may, for example, be labelled with a detectable molecule, or its binding to the cell detected by a secondary, labelled antibody, so that, after the incubation, it is possible to determine a first signal reflecting the number of binding molecules bound to the first cell or cell preparation and a second signal reflecting the number of binding molecules bound to the second cell or cell preparation. In some embodiments, the ratio between the first and second signal is at least 2:1, such as at least 3:1, such as at least 4:1, such as at least 5:1, such as at least 10:1. Alternatively, the ability of the binding molecule to bind to a nNav1.5 and an aNav1.5 can be determined in an in silico assay where the respective protein is bound to a solid support. Methods for determining the dissociation constant (KD) reflecting the binding affinity of an antibody to an antigen or other protein are well known in the art.

The binding molecule may also be characterized by specific VH, VL and/or CDR amino acid sequences, such as specific VH, VL and CDR amino acid sequences of monoclonal antibodies produced by clones or subclones described in Example 1.

Accordingly, in certain embodiments, the binding molecule comprises
(a) VH CDRs 1, 2 and 3 and VL CDRs 1, 2 and 3 of clone A3;
(b) VH CDRs 1, 2 and 3 and VL CDRs 1, 2 and 3 of subclone A3-2
(c) VH CDRs 1, 2 and 3 and VL CDRs 1, 2 and 3 of subclone C2-2, or
(d) VH and VL domains of clone A3.

In some embodiments, the binding molecule comprises a VH region comprising a complementary-determining region (CDR) 3 comprising SEQ ID NO:16, or a variant thereof comprising 1, 2 or 3 mutations; and a variable light chain (VL) region comprising a CDR3 comprising SEQ ID NO:19, or a variant thereof comprising 1, 2 or 3 mutations. The mutations may include, for example, one or more of substitutions, deletions and insertions of amino acids. In some embodiments, one, two or all mutations are amino acid substitutions, such as conservative amino acid substitutions.

In some embodiments, VH region further comprises a CDR1 comprising SEQ ID NO:14, or a variant thereof comprising 1, 2 or 3 mutations; and a CDR2 comprising SEQ ID NO:15, or a variant thereof comprising 1, 2 or 3 mutations; and the VL region comprises a CDR1 comprising SEQ ID NO:17, or a variant thereof comprising 1, 2 or 3 mutations; and a CDR2 comprising SEQ ID NO:18, or a variant thereof comprising 1, 2 or 3 mutations. The mutations may include, for example, one or more of substitutions, deletions and insertions of amino acids. In some embodiments, one, two or all mutations are amino acid substitutions, such as conservative amino acid substitutions.

In some embodiments, the binding molecule comprises a VH region comprising the CDR1, CDR2 and CDR3 of the VH region encoded by SEQ ID NO:10 and a VL region comprising the CDR1, CDR2 and CDR3 of the VH region encoded by SEQ ID NO:11.

In some embodiments, the binding molecule comprises a VH region comprising CDR1, CDR2 and CDR3 amino acid sequences comprising SEQ ID NOS:14, 15 and 16, respectively, and a VH region comprising CDR1, CDR2 and CDR3 amino acid sequences comprising SEQ ID NOS:17, 18 and 19, respectively.

In some embodiments, the binding molecule comprises a VH region at least 80%, such as at least 90%, 95%, 97%, 98% or 99% identical, to the VH region encoded by SEQ ID NO:10, and a VL region at least 80%, such as at least 90%, 95%, 97%, 98% or 99% identical, to the VL region encoded by SEQ ID NO:11.

In a specific embodiment, the binding molecule comprises a VH region comprising the amino acid sequence encoded by SEQ ID NO:10 and a VL region comprising the amino acid sequence encoded by SEQ ID NO:11.

In another specific embodiment, the binding molecule comprises a VH region comprising the amino acid sequence of SEQ ID NO:12 and a VL region comprising the amino acid sequence of SEQ ID NO:13.

In some embodiments, the binding molecule is an antibody comprising the antigen-binding sequences, particularly the VH and VL region sequences, of antibody (clone) A3 as described in Example 1.

Also provided is a binding molecule, such as a monoclonal antibody, antigen-binding fragment or CAR, which competitively inhibits a reference monoclonal antibody comprising the heavy chain and light chain of any one of clone A3, subclone A3-2 and subclone C2-2, such as the heavy chain and light chain of antibody (clone) A3, from specifically binding to nNaᵥ1.5, e.g., having the amino acid sequence of SEQ ID NO:2. A binding molecule is said to competitively inhibit binding of such a reference antibody to nNav1.5 if its presence reduces the binding of the reference antibody to the nNav1.5. Competitive inhibition may be determined by any method known in the art, for example, competition ELISA assays. A binding molecule may be said to competitively inhibit binding of the reference antibody to a given epitope by at least 90%, at least 80%, at least 70%, at least 60%, or at least 50%.

Further provided is a monoclonal antibody, antigen-binding fragment or CAR which specifically binds to the same epitope on a nNaᵥ1.5 as a monoclonal antibody comprising the heavy chain and light chain of any one of clone A3, subclone A3-2 and subclone C2-2, such as the heavy chain and light chain of antibody (clone) A3. The epitope may, for example, be a conformational epitope comprising at least 1, 2, 3, 4, 5 or more amino acid residues of exon 6 (SEQ ID NO. 3). The epitope may also be a linear epitope comprising at least 5, 6, 7, 8, 9, 10, 11, 12, 13, 14, 15 or more consecutive amino acids of SEQ ID NO:3. Preferably, the linear or conformational epitope in nNav1.5 comprises one, two, three, four, five, six or all of the amino acid residues V, S, N, I, K, L, and P in positions 206, 207, 209, 210, 211, 215, and 234, respectively. In some embodiments, the nNav1.5 has the amino acid sequence of SEQ ID NO:2. Methods for determining the epitope of an antibody are well-known in the art, and include, for example, X-ray co-crystallography and cryogenic electron microscopy (cryo-EM), array-based oligo-peptide scanning, site-directed mutagenesis mapping (e.g., alanine-scanning), high-throughput shotgun mutagenesis epitope mapping, hydrogen-deuterium exchange (HDX), and cross-linking-coupled mass spectrometry.

Also provided is a chimeric antigen receptor (CAR) comprising a binding portion which specifically binds to nNaᵥ1.5, a transmembrane domain and an endodomain. The binding portion preferably comprises an antigen-binding fragment of a binding molecule, such as a monoclonal antibody, according to any aspect or embodiment herein.

In some embodiments, at least one of the CAR, the monoclonal antibody and the antigen-binding fragment binds nNaᵥ1.5 more readily than it binds to adult 3′-exon splice variants of the α subunit of Naᵥ1.5 (aNaᵥ1.5), optionally with at least 100 times higher binding affinity compared to binding to aNav1.5. In one embodiment, the nNaᵥ1.5 comprises an amino acid sequence that differs from SEQ ID NO:1 in at least amino acid residue 211, such as in amino acid residues 206, 207, 209, 210, 211, 215, and 234. In one embodiment, the nNaᵥ1.5 comprises amino acid residues V, S, N, I, K, L, and P in positions 206, 207, 209, 210, 211, 215, and 234 respectively, and where aNaᵥ1.5 comprises the amino acid residues T, T, F, V, D, V, and S in the same/corresponding positions. In one embodiment, the nNaᵥ1.5 is human nNaᵥ1.5. In one embodiment, the CAR binds to an epitope in nNaᵥ1.5, which has the residue Lys (K) in position 211.

In some embodiments, there is provided a T cell or NK cell comprising a CAR according to any aspect or embodiment herein. Also provided is a nucleic acid sequence encoding the monoclonal antibody, antigen-binding fragment or CAR of any aspect or embodiment herein, an expression vector comprising the nucleic acid sequence, and a T cell or NK cell comprising said nucleic acid sequence or said expression vector.

In some embodiments, a binding molecule according to any aspect or embodiment herein is neutralizing. A “neutralizing” binding molecule, as used herein, is intended to refer to a binding molecule whose binding to nNav1.5 results in a reduction or inhibition of at least one biological activity of nNav1.5 or associated with nNav1.5. This reduction or inhibition of the biological activity of Nav1.5 can be assessed by measuring one or more indicators of Nav1.5 biological activity by one or more standard in vitro or in vivo assays known in the art.

For example, electrophysiological assays can be used for determining functional nNav1.5 ion conductance, in the case of Chioni et al. (2005) for evaluating the effect of a polyclonal antibody preparation on functional nNav1.5 ion conductance. Accordingly, the patch-clamp recording assay set out on page 91, section 2.8 of Chioni et al. (2005), hereby specifically incorporated by reference, can be used to evaluate whether a binding molecule as described herein is a neutralizing binding molecule. The effect of a binding molecule on the function of Nav1.5 can also be studied using the following electrophysiological assay, with details as described previously (e.g. Laniado et al., 1997; 2001; Fraser et al., 2003; Grimes et al, 1995), using, for example, nNav1.5 expressing EBNA cells prepared as described in Chioni et al. (2005):

Patch pipettes (tip resistances, ~5 MΩ) can be filled with a solution designed to block the outward K⁺ currents; e.g., (in mM): NaCl 5, CsCl 145, MgCl₂ 2, CaCl₂) 1, HEPES 10 and EGTA 11, adjusted to pH 7.4 with 1 M CsOH. The intracellular free Ca²⁺ concentration can be estimated, e.g., ~15 nM (Laniado et al., 2001). Whole-cell membrane currents can be recorded from cells that appear ‘isolated’ in culture, e.g., using an Axopatch 200B amplifier (Axon Instruments, CA, USA). Analogue signals can be filtered at 10 kHz using a low-pass Bessel filter, and series resistance errors can be compensated by >90%. Electrophysiological signals may be sampled at 50 kHz and digitized, e.g., using an interface such as Digidata 1200. Data acquisition and analysis of whole-cell currents can then be performed, e.g., using suitable software such as pClamp software (Axon Instruments). A holding potential of −100 mV may be applied. Standard voltage-clamp protocols were used to study the electrophysiological properties of the VGSC currents. All routine recordings can be done after a suitable time of incubation, e.g., at 24 hours after (re)plating and 24 hours of serum starvation (to match the condition of the invasion assays). Conductance-voltage relationships and other relevant parameters for evaluating VGSC currents can then be calculated using equations (I) to (III) below, with further details provided in Onkal et al. (2008):

Conductance-voltage relationships are determined using the equation:

G=I/(V−Vᵣₑᵥ) (I),

where G is the conductance; I the current amplitude; V the test pulse; and Vᵣₑᵥ the theoretical Na⁺ reversal potential. Normalized curves for voltage dependence of steady-state activation and inactivation are fitted to a Boltzmann function of the form:

G=Gₘₐₓ/[1−exp(Vₘ−V_{1/2})/k] (II)

where Gₘₐₓ is the maximal conductance; Vₘ is the membrane voltage; V_{1/2} is the voltage at which the current is half activated/inactivated, and k is the slope factor of voltage sensitivity. For the time course of recovery from inactivation, the data is plotted as a function of recovery time and fitted to the following single exponential equation:

I=A exp(−t/t)+C (III)

where I is normalized current, t is time, t is the time constant, A is the amplitude of the normalized current, and C is the asymptote. More details can be found in Onkal et al. (2008).

Typically, a neutralizing binding molecule according to the present invention results in a significant reduction in VGSC current, e.g. peak VGSC current density and/or absolute current, relative to control and/or significantly reduces the proportion of cells demonstrating VGSC currents, e.g., by at least about 10%, 20% or more, such as by at least 30%, 40%, 50%, 60%, 70% or more as compared to a control.

In one particular embodiment, the VGSC comprises a nNav1.5 α subunit, and a neutralizing binding molecule according to the present invention results in a significant reduction in VGSC current, e.g. peak VGSC current density and/or absolute current, relative to control and/or significantly reduces the proportion of cells demonstrating nNav1.5 currents, e.g., by at least about 10%, 20% or more, such as by at least 30%, 40%, 50%, 60%, 70% or more as compared to a control.

In one particular embodiment, and a neutralizing binding molecule according to the present invention results in a reduction in VGSC current, e.g., peak VGSC current density and/or absolute current, of a VGSC comprising a nNav1.5 α subunit which is higher than the corresponding reduction in VGSC current of a VGSC comprising an aNav1.5 α subunit, e.g., by at least about 10%, 20% or more, such as by at least 30%, 40%, 50%, 60%, 70% or more.

Additionally, the neutralizing ability of a binding molecule to reduce the invasiveness of cancer cells can be determined using the assay described by Fraser et al. (2005) and/or Brackenbury et al. (2007), following optimization of the cell number versus the Matrigel concentration. Subsequently, (i) insert filters (with 8 μm pores) can be coated with 50 μl of 0.21 mg/ml Matrigel (BD Biosciences, Bedford, MA, USA); (ii) a chemotactic gradient can be 0.1-10% FBS; and (iii) the cells can be serum-starved for 24 hours and (iv) about 10⁵ cells can be seeded onto each filter. After a suitable period of incubation, such as 48 hours, the insert can be swabbed and then stained with crystal violet. The invaded cells in 12 non-overlapping fields of view can then be counted, e.g., under ×400 magnification. “Invasiveness” can then be calculated as the number of invaded cells normalized to the largest value observed amongst the different treatment conditions in given experimental sets. Typically, a neutralizing binding molecule of the present invention results in a significant reduction of invasiveness, e.g., by at least about 10%, 20% or more, such as by at least 30%, 40%, 50%, 60%, 70% or 80% as compared to a control. This may be observed both under normoxic and hypoxic conditions.

Suitable controls include, for example, the buffer or other solution in which the binding molecule is dissolved and a control binding molecule whose VH and VL regions specifically bind to an irrelevant antigen but the remainder of the control binding molecule is the same as the test binding molecule. For example, in the case of a binding molecule which is a test monoclonal antibody or test antigen-binding fragment, a control monoclonal antibody or antigen-binding fragment specifically binding to an irrelevant antigen but having the same framework and/or constant regions as the test monoclonal antibody or test antigen-binding fragment can be used.

Also contemplated are binding molecules conjugated to cytotoxic agent, radioisotope, chemotherapeutic drug or the like, e.g., in the form of an antibody-drug conjugate (ADC), which delivers a cytotoxic agent or chemotherapeutic drug to nNav1.5-expressing tumor cells by way of the binding specificity of the binding molecule. In some embodiments, an ADC according to the invention is used to target micrometastases. nNav1.5-based ADCs could offer a particular advantage of recognizing and killing micro-metastases, one of the most difficult problems in clinical management of cancer, since they can be “dormant” and thereby difficult to both detect and treat by conventional means. ADCs are often designed such that the cytotoxic payload is inactive when conjugated to the antibody. The cytotoxic payload may be released intracellularly upon internalization of the ADC after binding to the plasma-membrane of cells, or alternatively in response to proteolytic activity in the tumor microenvironment. This can, for example, be accomplished by way of the binding molecule being conjugated to a cytotoxic agent or chemotherapeutic drug via a cleavable linker, which is cleaved by e.g. an intracellular peptidase or protease or upon internalization of the ADC, or by proteolytic enzymes in the tumor microenvironment. Non-limiting examples of cytotoxic agents include, for example, auristatins, maytansinoids, calicheamicins and amatoxins. Further examples include DNA-targeting agents, e.g. DNA alkylators and cross-linkers, such as calicheamicin, duocarmycin, rachelmycin (CC-1065), pyrrolo[2,1-c][1,4]benzodiazepines (PBDs), and indolinobenzodiazepine (IGN); microtubule-targeting agents, such as duostatin, such as duostatin-3, auristatin, such as monomethylauristatin E (MMAE) and monomethylauristatin F (M MAF), dolastatin, maytansine, /V(2′)-deacetyl-/V(2′)-(3-marcapto-l-oxopropyl)-maytansine (DM1), and tubulysin; and nucleoside analogs; as well as analogs, derivatives, or prodrugs thereof. Examples of chemotherapeutic drugs include microtubule-targeting agents such as paclitaxel, docetaxel, vinblastine, vincristine, vinorelbine, duostatins, auristatins, maytansanoids, tubulysins, and dolastatin.

Further contemplated is a multispecific, such as a bispecific, antibody which comprises a first binding domain and a second binding domain, wherein the first and second binding domains have different binding specificities. In one embodiment, the first binding domain binds to nNav1.5 and the second binding domain binds to a second antigen which is not nNav1.5. For example, Nav1.5 has been shown to be physically associated with sodium-hydrogen exchanger protein (NHE1) (Brisson et al., 2013). So, in one embodiment, there is provided a multispecific, such as a bispecific, antibody comprising a first binding domain which specifically binds to nNav1.5 according to any aspect or embodiment herein, and a second binding domain which specifically binds to NHE1. The first binding domain may, for example, have the binding specificity of the monoclonal antibody produced by clone A3, subclone A3-2, or subclone C2-2. Preferably, the first binding domain comprises VH and VL regions comprising the CDRs, VH and/or VL region sequences of antibody (clone) A3, as described in Example 1 and the Sequence Table.

In another embodiment there is provided a multispecific antibody, such as a bispecific antibody, wherein a first binding domain binds to nNav1.5 and a second binding domain which specifically binds to the voltage-dependent ion channel Kv 11.1, also known as hERG1 (the “human Ether-à-go-go-Related Gene”) and encoded by the gene KCNH2 (Duranti and Arcangeli. Ion Channel Targeting with Antibodies and Antibody Fragments for Cancer Diagnosis. Antibodies (Basel). 2019 May 24; 8(2):33).

A bispecific or multispecific antibody may be particularly useful for treating a cancer expressing both nNav1.5 and NHE1, or both nNav1.5 and hERG1. In a particular embodiment, the cancer is a breast cancer.

In another embodiment there is provided a multispecific antibody, such as a bispecific antibody, wherein the first and second binding domains specifically bind to different epitopes on nNav1.5. For example, in one embodiment, the first binding domain may have the binding specificity of the monoclonal antibody produced by clone A3 or A3-2, and the second binding domain may have the binding specificity of the monoclonal antibody produced by subclone C2-2, or vice versa. Preferably, the first binding domain comprises VH and VL regions comprising the CDRs, VH and/or VL region sequences of antibody (clone) A3, as described in Example 1.

### Production of Binding Molecules

The disclosure further provides polynucleotides, e.g., an isolated, recombinant, and/or non-naturally occurring polynucleotide, comprising a nucleic acid sequence that encodes at least a subunit of the binding molecule specifically binding to nNav1.5 according to any aspect or embodiment herein. By “polypeptide subunit” is meant a portion of a binding molecule, or antigen binding domain that can be independently translated. Examples include, without limitation, an antibody variable domain, e.g., a VH or a VL region, an antibody heavy chain, an antibody light chain, a single-chain Fv (scFv) antibody comprising a VH or a VL region, a CAR comprising such an scFv, and/or any fragment, variant, or derivative thereof.

In certain aspects, the subunit can comprise an IgG (such as an IgG1, IgG2, IgG3 or IgG4), IgA or IgM heavy chain constant region or fragment thereof, and VH region of a binding molecule which specifically binds to nNav1.5. In certain aspects the nucleic acid sequence can encode α subunit comprising a human IgG constant region or fragment thereof fused to the C-terminal end of a VH region, where the VH region comprises the VH CDR1, CDR2, and CDR3 of the VH region of antibody (clone) A3, or a variant thereof comprising 1, 2 or 3 mutations, such as amino acid substitutions. In some embodiments, the nucleic acid sequence encodes a subunit comprising a human IgG constant region or fragment thereof, such as an IgG1 or IgG4 constant region or fragment thereof, fused to the C-terminal of the amino acid sequence encoded by SEQ ID NO:10, or a variant thereof as described elsewhere herein.

In certain aspects, the subunit can comprise an antibody VL portion of a binding molecule which specifically binds to nNav1.5 as described above. In certain aspects the nucleic acid sequence can encode a subunit comprising a human antibody light chain constant region or fragment thereof fused to the C-terminal end of a VL region, wherein the VL region comprises the VL CDR1, CDR2, and CDR3 of the VL region of antibody (clone) A3, or a variant thereof comprising 1, 2 or 3 mutations, such as amino acid substitutions. In some embodiments, the nucleic acid sequence encodes a subunit comprising a human light chain constant region, e.g., a human kappa or lambda constant light chain region or fragment thereof, fused to the C-terminal of the amino acid sequence encoded by SEQ ID NO:11, or a variant thereof as described elsewhere herein.

The disclosure further provides a composition comprising two (or more) polynucleotides encoding different subunits, e.g., an antibody heavy and a light chain, respectively, which collectively can encode a binding molecule which specifically binds to nNav1.5 as described above. In certain aspects, the two polynucleotides making up the composition can be situated on a single vector, e.g., an expression vector. Vectors useful for these purposes are known in the art. Such vectors can also comprise enhancer and other sequences needed to achieve expression of the desired chains.

Further provided are cells, e.g., a hybridoma or a host cell, such as, e.g., a prokaryotic or eukaryotic host cell, comprising a polynucleotide or two or more polynucleotides encoding a binding molecule as provided herein, or any subunit thereof, a polynucleotide composition as provided herein, or a vector or two, three, or more vectors that collectively encode a binding molecule as provided herein, or any subunit thereof. The host cell can, for example, be described as a recombinant host cell. In certain aspects a recombinant host cell provided by the disclosure can express a binding molecule which specifically binds to nNav1.5, or a subunit thereof.

To form the antigen binding domains, the variable regions of antibodies that specifically bind to nNav1.5 can be inserted into expression vector templates for antibodies, e.g., human IgG, IgA or IgM antibodies, thereby creating multimeric binding molecules having at least two bivalent binding units. In brief, nucleic acid sequences encoding the heavy and light chain variable domain sequences can be synthesized or amplified from existing molecules, and inserted into vectors in the proper orientation and in frame such that upon expression, the vector will yield a full length heavy or light chain, or a desired fragment thereof. Multiple vectors or single vectors can be used. These vectors are transfected into host cells and the polypeptide chain(s) thereafter expressed and optionally purified.

In a related aspect, the disclosure provides a method of producing a binding molecule specifically binding to nNav1.5 as provided by this disclosure, where the method comprises culturing a host cell as described above and, optionally, recovering the binding molecule.

In some embodiments, particularly when the binding molecule is CAR, a polynucleotide as described herein may further comprise nucleic acid sequences encoding the other regions typically present in a CAR molecule. For example, the polynucleotide may encode a polypeptide comprising nNav1.5-binding domain/hinge/transmembrane domain/endodomain regions, e.g., wherein the nNav1.5-binding domain may be a scFv, the hinge may comprise an IgG1 or IgG4 hinge region, and the transmembrane domain and the most membrane-proximal molecule of the endodomain may derive from the same molecule. The endodomain may, for example, comprise, from membrane-proximal to membrane-distal, CD3-zeta, CD28/CD3-zeta, or CD28/4-IBB/CD3-zeta.

### Therapeutic and Diagnostic Methods

A binding molecule according to any aspect or embodiment described herein, a nucleic acid or vector encoding such a binding molecule, or a cell expressing such a binding molecule, may be used as a medicament, e.g., in methods for treatment or amelioration of cancer, in methods for reducing the risk of metastatic cancer, or both.

A binding molecule according to any aspect or embodiment described herein may also be used as a nNav1.5 detection agent in in vivo or in vitro methods, e.g., to detect nNav1.5 expressing tumours, metastases or cancer cells.

In one embodiment, the binding molecule is a monoclonal antibody which specifically binds to nNaᵥ1.5. In one embodiment, the binding molecule is an antigen-binding fragment of a monoclonal antibody which specifically binds to nNaᵥ1.5. In one embodiment, the binding molecule is a CAR which specifically binds to nNaᵥ1.5, optionally wherein the CAR is comprised in an NK or T cell. Depending on the intended use of the binding molecule, the binding molecule can further be, for example, neutralizing, conjugated to a cytotoxic drug (e.g., as an antibody-drug conjugate (ADC)), or labelled with a detectable label.

In one aspect, the method is for treatment or amelioration of metastatic cancer, comprising administering an effective amount of a binding molecule according to the invention to a patient suffering from metastatic cancer.

In one aspect, the method is for reducing the risk of metastatic cancer in a subject, comprising administering an effective amount of a binding molecule according to the invention to said subject.

In one aspect, the method is a method for detecting the presence of metastatic cancer in a subject, the method comprising administering a labelled antibody or antigen-binding fragment thereof, which specifically recognizes nNaᵥ1.5, to the subject and subsequently measuring signal distribution derived from the labelled antibody or antigen-binding fragment in said subject, where a localised dense signal in a part of the body of the subject is indicative of the presence of metastatic disease in said part of the body.

In one aspect, the method is for detecting the presence of nNav1.5 in a biological sample from a subject, the method comprising contacting the biological sample with an antibody or antigen-binding fragment thereof which specifically recognizes nNaᵥ1.5, and subsequently detecting binding of the antibody or antigen-binding fragment to the biological sample.

VGSC is a mammalian gene family. Accordingly, suitable subjects or patients include mammalian subjects or patients, such as humans, monkeys, rabbits, dogs, cats, cows, horses, pigs, mice and rats, suffering from cancer. Preferably, the patient is a human patient, such as an adult human patient. For example, in one embodiment, the subject has a high risk of developing metastatic cancer, such as a patient having a familiar disposition for metastatic cancer. In one embodiment, the subject is a cancer patient who has undergone surgical, medical, and/or radiation anti-cancer therapy without showing clinical signs of metastatic disease.

The cancer can be selected from solid tumours and non-solid tumours. Preferably, the cancer is a nNav1.5-expressing cancer or known to be associated with a risk for increased nNav1.5 expression, metastatic behaviour, invasiveness, aggressiveness, or any combination thereof. In some embodiments, the cancer is a carcinoma. In some embodiments, the cancer is a cancer of a tissue selected from the lung, prostate, stomach, breast, large intestine, rectum, ovary, pancreas, liver, and CNS, such as the brain. In a particular embodiment, the cancer is cancer of the breast or colon or ovary. In another particular embodiment, the cancer is selected from the group consisting of colorectal cancer, breast cancer, lung cancer, ovarian cancer, neuroblastoma and astrocytoma, or a combination of any thereof. Preferably, the cancer is a VGSC-expressing cancer. In some embodiments, the cancer expresses a nNav1.5.

As used herein, “treating” or “treatment” of a cancer includes, but is not limited to, reducing metastatic behaviour of a cancer, reducing pain sensation, preventing metastatic behaviour of a cancer, reducing pain sensation, reducing the invasiveness of a cancer, reducing the overall aggressiveness of the cancer, or any combination thereof. So, in separate and specific embodiments, a method of treatment according to the invention may (i) reduce metastatic behaviour of the cancer, (ii) prevent metastatic behaviour of the cancer, (iii) reduce pain sensation in a patient suffering from the cancer, (iv) reduce the invasiveness of the cancer, or (v), and combination of two or more of (i) to (iv).

Progression of metastatic cancer, such as breast, colon and prostate cancer, is generally considered as comprising at least some of five main phases, as follows:
1. Genesis, namely the initial transformation of a normal cell into a cancer cell;
2. Proliferation, namely increase in the number of cancer cells to form a primary tumour of increasing size, typically with a smooth and well defined surface;
3. Switching, during the genesis or proliferation phase, from a condition in which the cancer cells have no potential for invasive or metastatic behaviour to a condition in which they do, typically characterised by a dissolving and diffuse boundary of the cancer;
4. Detachment of cancer cells from the primary tumour followed by movement of those detached cells into surrounding regions of tissue within the same organ towards the circulation system (blood and/or lymph);
5. Metastasis, namely the movement of the detached cells through the circulation (blood or lymph) to other organs to create secondary tumours in those other organs.

It should be noted, however, that metastasis may occur without an initial proliferative phase. In such cases, metastases may be found in a patient without an identifiable primary tumour.

By “reducing metastatic behaviour” of cancer, it is intended a reduction of any behaviour associated with the movement of detached cancer cells through the circulation (blood or lymph) to accumulate and/or create secondary tumours in other organs or locally invade surrounding tissues. Typically, the patient is in phase 3, 4 or 5, such as in phase 4 or 5. Reducing metastatic behaviour may, for example, include one or more of (i) reducing transcription, translation and/or expression of nNav1.5 in cancer cells as compared to a control; (ii) reducing cancer cell invasiveness; (iii) reduce peak VGSC current density in cancer cells; (iv) reduce the proportion of cancer cells demonstrating VGSC currents; (v) reducing cancer cell motility (e.g., reduced lateral motility), (vi) reducing cancer cell migration (e.g., transverse migration), and (vii) reducing the persistent part of the VGSC current without eliminating the transient part. “Motility” reflects the ability of the tumour cells to initially move to and through the basement membrane into the surrounding tissue; “invasiveness” of the cells reflects the ability of tumour cells which have entered the surrounding tissue to move through that tissue towards the circulation system; and “migration” reflects the ability of the tumour cells to migrate from that tissue into the circulatory system via the walls thereof.

By “reducing the risk for metastatic cancer” or “preventing metastatic behaviour” of cancer, it is intended to refer prophylactic treatment of a cancer patient at risk for, but not yet diagnosed with, a metastatic disease, so as to prevent or reduce the risk for a metastatic behaviour of the cancer as described above. Typically, the patient is in phase 1, 2 or 3.

The term “benign state” as used herein refers to a tumour or cancer in phase 1 or 2. As used herein, tumours may also or alternatively be characterized as being in a benign state if they (a) do not invade nearby tissue (invasiveness); (b) do not metastasize (spread) to other parts of the body; (c) tend to have clear boundaries; and/or (d) grow slowly.

The term “malignant state” herein refers to a tumour or cancer in phase 3, 4 or 5.

By “reducing the overall aggressiveness of a cancer”, it is intended a reduction of any behaviour associated with the progression of cancer, in quantitative or qualitative terms. In some embodiments, reducing the aggressiveness of a cancer refers to the reversal of a cancer in any one of phase 3, 4 or 5 to a lower-number phase, including, but not limited to, from phase 3 to phase 2 or lower, from phase 4 to phase 3 or lower, and from phase 5 to phase 4 or lower. In some embodiments, reducing the aggressiveness of a cancer refers to the reversal of a cancer or tumour in a malignant state to a cancer or tumour in a benign state. In some embodiments, by “reducing the overall aggressiveness of a cancer”, it is intended a reduction of a cancer to non-metastatic but not necessarily non-invasive state.

By “reducing the invasiveness of a cancer”, it is intended a significant reduction of the invasiveness of the cancer cells under predetermined conditions, e.g., normoxic or hypoxic conditions. Examples of assays suitable to determine invasiveness are provided elsewhere herein (see, e.g., the section entitled “Functional characteristics”). A significant reduction of invasiveness includes, e.g., a reduction by at least about 10%, 20% or more, such as by at least 30%, 40%, 50%, 60%, 70% or 80% as compared to a control.

In some embodiments of the methods of the invention, the binding molecule is administered in a therapeutically effective amount or dose. By “therapeutically effective amount”, “therapeutically effective dose”, it is intended an amount or dosage of binding molecule that, when administered to a patient suffering from cancer brings about a positive therapeutic response with respect to treatment of the patient, such as, e.g., reduction of metastatic behaviour of the cancer, prevention of metastatic behaviour of the cancer, reduction of pain, or the like.

The binding molecule is administered to the patient in a therapeutically effective amount for the intended purpose, and with a frequency and for a period of time determined by a trained physician. Estimates of effective dosages and in vivo half-lives for the individual binding molecules encompassed by the invention can be made using conventional methodologies or on the basis of in vivo testing using an appropriate animal model.

The binding molecule can be formulated in a composition, typically a pharmaceutical composition, for administration by any suitable route to the patient, including, but not limited to, oral, buccal, sublabial, sublingual, rectal, intravenous, subcutaneous, intradermal, intramuscular, transdermal and intranasal administration and/or direct administration to a tumour, such as a primary tumour. Delivery can, for example, be performed by systemic administration or local administration (e.g., to a tumor), of a composition, such as a pharmaceutical composition, comprising the binding molecule to a subject, e.g., a cancer patient. In some embodiments, the pharmaceutical composition is administered via injection or infusion to the subject, e.g., a cancer patient. In some embodiments, the composition is administered by direct administration into a tumor mass.

In some embodiments, one or more tumours in the patient are hypoxic or are at risk for being hypoxic. In some embodiments, one or more tumours in the patient are expected or determined by the trained physician to be hypoxic. The presence of hypoxia can be determined by a variety of techniques known in the art, including, but not limited to, magnet resonance (MR) imaging (see, e.g., Abadjian et al., Adv Exp Med Biol. 2017; 1036:229-257) or staining a sample of tumour tissue with pimonidazole (see, e.g., Wilson and Hay, Nature Reviews Cancer 2011; 11: 393-410).

In some embodiments, the patient is suffering from a cancer comprising nNav1.5-expressing cancer cells. Such a cancer may, for example, be identified by immunohistochemical or analysis of a cancer cell-containing sample (such as a tumour biopsy or blood sample) obtained from the patient, using detectable monoclonal or polyclonal antibodies specific for nNav1.5 to detect the expression of Nav1.5 by the cancer cells. Advantageously, a detectable monoclonal antibody or antigen-binding fragment thereof can be used for this purpose.

In some embodiments, the treatment methods described herein comprises a step of determining that the cancer comprises cells expressing nNav1.5, typically conducted prior to administering the binding molecule. This can be performed by taking a sample from a tumour in the subject contemplated for treatment, e.g., a tumour biopsy, and analysing the tumour sample or tumour cells for the relevant nNav1.5 mRNA, expression of nNav1.5 protein, or both. Suitable assays for doing this are described elsewhere herein. In a preferred embodiment, the treatment method comprises a step of determining that the cancer comprises cells expressing nNav1.5 prior to administering the binding molecule.

In some embodiments, the therapeutic use or method according to any aspect or embodiment herein is in combination with a second therapeutic agent. In some embodiments, said second therapeutic agent is ranolazine or eleclazine. Suitable dosages of ranolazine and eleclazine can be found in WO2012/049440 (Celex Oncology Ltd.) and WO 2018/146313 (Celex GmbH), both of which are hereby incorporated by reference in their entireties. In some embodiments, the cancer comprises a hypoxic tumor.

In one embodiment, a nNav1.5-expressing cancer is in phase 3, 4 or 5 as described above.

In one embodiment, the patient is in phase 3, 4 or 5, such as in phase 4 or 5.

In one embodiment, the cancer is in phase 1, 2, or 3, such as in phase 1 or 2.

In one embodiment, the cancer is in phase 3. A patient suffering from a cancer in phase 3 has typically not been diagnosed with metastatic disease, but is at risk for metastatic behaviour of the cancer, i.e., progression to phase 4 or 5. A patient suffering from a cancer in phase 3 may thus be treated according to the invention to reduce the risk for metastatic behaviour of the cancer.

In one embodiment, the cancer is in phase 4. A patient suffering from a cancer in phase 4 may not have been diagnosed with metastatic disease, but the cancer has progressed towards metastatic behaviour. A patient suffering from a cancer in phase 4 may thus be treated according to the invention to reduce metastatic behaviour of the cancer.

In one embodiment, the cancer is in phase 5. A patient suffering from a cancer in phase 5 may have been diagnosed with metastatic disease, and the cancer is characterized by metastatic behaviour. A patient suffering from a cancer in phase 5 may thus be treated according to the invention to reduce metastatic behaviour of the cancer.

In some embodiments, a patient may be suffering from a cancer associated with a risk for nNav1.5-expression and/or metastasis or metastatic behaviour, but nNav1.5-expression and/or metastatic behaviour has not yet been determined. Cancers that are prone to metastatic behaviour include, for example, colon cancer, breast cancer, lung cancer, prostate cancer and ovarian cancer. For example, an in vivo diagnostic method according to the invention may not have detected a localised dense signal in a part of the body of the subject indicative of the presence of metastatic disease in said part of the body. Alternatively, immunohistochemical analysis of a biological sample, such as a cancer cell-containing sample, e.g., a tumour biopsy or blood sample obtained from the patient, may not have indicated that the tumour cells in the sample express nNav1.5. The cancer may thus be in phase 1 or (more likely) in phase 2.

In one embodiment, the cancer is in phase 2. A patient suffering from a cancer in phase 2 has typically not been diagnosed with metastatic disease, but is at risk for nNav1.5 expression and metastatic behaviour of the cancer, i.e., progression to phase 3, 4 or higher. A patient suffering from a cancer in phase 2 may thus be treated according to the invention to prevent nNav1.5-activity or metastatic behaviour of the cancer.

A patient suffering from a cancer in any one of phase 1-5, such as in any one of 2-5, may also suffer from pain caused by the cancer, e.g., by a primary tumour, and may thus be treated according to the invention to reduce pain sensation.

In one embodiment, when used in a method according to the invention, the binding molecule reduces or prevents metastatic behaviour in nNav1.5-expressing cancer without killing the cancer cells.

In one embodiment, treatment of cancer cells with the binding molecule results in the VGSC activity of nNav1.5 of the cancer cell, e.g., the VGSC current, being significantly lower than that of a control, such as a predetermined control value, cancer cells not exposed to the binding molecule or cancer cells exposed to a reference compound, e.g., an antibody against an irrelevant antigen. In one embodiment, treatment of cancer cells with the binding molecule results in the invasiveness, motility and/or ability to migrate of cancer cells treated with the binding molecule being significantly lower than that of a control, such as a predetermined control value, cancer cells not exposed to the binding molecule or cancer cells exposed to a selected reference compound.

### FURTHER EMBODIMENTS

The following are further specific embodiments according to the invention.
1. A monoclonal antibody which specifically binds to neonatal 5′-exon splice variants of the α subunit of Naᵥ1.5 (nNaᵥ1.5).
2. The monoclonal antibody according to embodiment 1, which binds nNaᵥ1.5 with at least 100 times higher binding affinity compared to binding to adult 3′-exon splice variants of the α subunit of Naᵥ1.5 (aNaᵥ1.5).
3. The monoclonal antibody according to embodiment 1 or 2, wherein the nNaᵥ1.5 comprises an amino acid sequence that differs from SEQ ID NO: 1 in at least amino acid residue 211, such as in amino acid residues 206, 207, 209, 210, 211, 215, and 234.
4. The monoclonal antibody according to embodiment 3, wherein the nNaᵥ1.5 comprises amino acid residues V, S, N, I, K, L, and P in positions 206, 207, 209, 210, 211, 215, and 234 respectively, and where aNaᵥ1.5 comprises the amino acid residues T, T, F, V, D, V, and S in the same/corresponding positions.
5. The monoclonal antibody according to any one of the preceding embodiments, where the nNaᵥ1.5 is human nNaᵥ1.5.
6. The monoclonal antibody according to any one of the preceding embodiments, which binds to an epitope in nNaᵥ1.5, which has the residue Lys (K) in position 211.
7. The monoclonal antibody according to any one of the preceding embodiments, where all constant domains, framework regions, or constant and framework regions, are human.
8. The monoclonal antibody according to embodiment 7, which is chimeric, humanized, or fully human.
9. An antigen-binding fragment of the monoclonal antibody according to embodiment 8.
10. A chimeric antigen receptor (CAR) comprising a binding portion which specifically binds to neonatal 5′-exon splice variants of the α subunit of Naᵥ1.5 (nNaᵥ1.5), a transmembrane domain and an endodomain.
11. The CAR of embodiment 10, wherein the binding portion comprises an antigen-binding fragment of a monoclonal antibody.
12. The CAR of embodiment 11, wherein the monoclonal antibody is a monoclonal antibody according to any one of embodiments 1 to 8.
13. The CAR of any one of embodiments 11 and 12, wherein the antigen-binding fragment is a single-chain Fv (scFv) fragment or a Fab fragment.
14. The CAR according to any one of embodiments 10 to 13, wherein at least one of the CAR, the monoclonal antibody and the antigen-binding fragment binds nNaᵥ1.5 with at least 100 times higher binding affinity compared to binding to adult 3′-exon splice variants of the α subunit of Naᵥ1.5 (aNaᵥ1.5).
15. The CAR according to any one of embodiments 10 to 14, wherein the nNaᵥ1.5 comprises an amino acid sequence that differs from SEQ ID NO: 1 in at least amino acid residue 211, such as in amino acid residues 206, 207, 209, 210, 211, 215, and 234.
16. The CAR according to embodiment 15, wherein the nNaᵥ1.5 comprises amino acid residues V, S, N, I, K, L, and P in positions 206, 207, 209, 210, 211, 215, and 234 respectively, and where aNaᵥ1.5 comprises the amino acid residues T, T, F, V, D, V, and S in the same/corresponding positions.
17. The CAR according to any one of embodiments 10 to 16, where the nNaᵥ1.5 is human nNaᵥ1.5.
18. The CAR according to any one of embodiments 10 to 17, which binds to an epitope in nNaᵥ1.5, which has the residue Lys (K) in position 211.
19. The CAR according to any one of embodiments 10 to 18, wherein the endodomain comprises a signalling domain derived from a T cell receptor molecule, such as the CD3-ζ (CD3-zeta) chain.
20. The CAR according to embodiment 19, wherein the endodomain further comprises one or more co-stimulatory molecules comprising at least one of CD27, CD28, 4-IBB and OX40.
21. The CAR according to any one of embodiments 10 to 20, wherein the transmembrane domain and the most membrane-proximal portion of the endodomain are derived from the same molecule.
22. The CAR according to any one of embodiments 10 to 21, comprising a linker peptide fused to the binding domain and the transmembrane domain.
23. The CAR according to embodiment 10 to 22, wherein the linker peptide comprises the hinge portion of the constant region of an immunoglobulin, such as IgG1 or IgG4.
24. The monoclonal antibody of any one of embodiments 1 to 8, the antigen-binding fragment of embodiment 9, or the CAR of any one of embodiments 10 to 23, which comprises
   (a) variable heavy (VH) complementary-determining regions (CDRs) 1, 2 and 3 and variable light (VL) CDRs 1, 2 and 3 of clone A3;
   (b) VH CDRs 1, 2 and 3 and VL CDRs 1, 2 and 3 of subclone A3-2; or
   (c) VH CDRs 1, 2 and 3 and VL CDRs 1, 2 and 3 of subclone C2-2.
25. The monoclonal antibody, antigen-binding fragment or CAR of embodiment 24, which comprises
   (a) a VH region at least 80%, such as at least 90%, 95%, 97%, 98% or 99% identical, to a VH of clone A3, and a VL region at least 80%, such as at least 90%, 95%, 97%, 98% or 99% identical, to a VL of clone A3;
   (b) a VH region at least 80%, such as at least 90%, 95%, 97%, 98% or 99% identical, to a VH of subclone A3-2, and a VL region at least 80%, such as at least 90%, 95%, 97%, 98% or 99% identical, to a VL of subclone A3-2; or
   (c) a VH region at least 80%, such as at least 90%, 95%, 97%, 98% or 99% identical, to a VH of subclone C2-2, and a VL region at least 80%, such as at least 90%, 95%, 97%, 98% or 99% identical, to a VL of subclone C2-2.
26. The monoclonal antibody, antigen-binding fragment or CAR of any one of embodiments 24 and 25, which comprises
   (a) a VH region comprising a VH of clone A3 and a VL region comprising a VL of clone A3;
   (b) a VH region comprising a VH of subclone A3-2 and a VL region comprising a VL of subclone A3-2;
   (c) a VH region comprising a VH of subclone C2-2 and a VL region comprising a VL of subclone C2-2; or
   (d) the heavy chain and light chain of any one of clone A3, subclone A3-2 and subclone C2-2.
27. A monoclonal antibody, antigen-binding fragment or CAR which competitively inhibits a monoclonal antibody according to embodiment 26 from specifically binding to neonatal 5′-exon splice variants of the α subunit of Naᵥ1.5 (nNaᵥ1.5).
28. A monoclonal antibody, antigen-binding fragment or CAR which specifically binds to the same epitope on neonatal 5′-exon splice variants of the α subunit of Naᵥ1.5 (nNaᵥ1.5) as a monoclonal antibody according to embodiment 26.
29. The monoclonal antibody, antigen-binding fragment or CAR according to any one of embodiments 1 to 28, which specifically binds to a nNav1.5 comprising the peptide VSENIKLGNLSALR, corresponding to residues 206 to 219 of SEQ ID NO:2.
30. The monoclonal antibody, antigen-binding fragment or CAR according to any one of embodiments 1 to 28, which specifically binds to a neonatal 5′-exon splice variants of the α subunit of Naᵥ1.5 (nNaᵥ1.5) comprising SEQ ID NO:2.
31. A bispecific antibody comprising a first antigen-binding domain which specifically binds to nNav1.5 and a second antigen-binding domain.
32. The bispecific antibody according to embodiment 24, wherein the first antigen-binding domain comprises the features of the monoclonal antibody according to any one of embodiments 1 to embodiments 1 to 9 or 24 to 31.
33. The bispecific antibody according to any one of embodiments 31 to 32, wherein the second antigen-binding domain specifically binds to sodium-hydrogen exchanger protein (NHE1).
34. A nucleic acid sequence encoding the monoclonal antibody, antigen-binding fragment, CAR, or bispecific antibody of any one of embodiments 1 to 33.
35. An expression vector comprising the nucleic acid sequence of embodiment 34.
36. A T cell or NK cell comprising the nucleic acid sequence of embodiment 34 or the expression vector of embodiment 35.
37. A T cell or NK cell comprising a CAR according to any one of embodiments 10 to 30.
38. A monoclonal antibody according to any one of embodiments 1-8 and 24-29, an antigen-binding fragment according to any one of embodiments 9 and 24 to 30, a bispecific antibody according to any one of embodiments 31 to 33, or a T cell or NK cell according to any one of embodiments 36 and 37, for use as a medicament.
39. A monoclonal antibody according to any one of embodiments 1-8 and 24-29, an antigen-binding fragment according to any one of embodiments 9 and 24 to 30, a bispecific antibody according to any one of embodiments 31 to 33, or a T cell or NK cell according to any one of embodiments 36 and 37, for use in treating cancer.
40. A method for treatment or amelioration of metastatic cancer, comprising administering an effective amount of a monoclonal antibody according to any one of embodiments 1-8 and 24-29, an antigen-binding fragment according to any one of embodiments 9 and 24 to 30, a bispecific antibody according to any one of embodiments 31 to 33, or a T cell or NK cell according to any one of embodiments 36 and 37, to a patient suffering from metastatic cancer.
41. A method for reducing the risk for metastatic cancer in a subject, comprising administering an effective amount of a monoclonal antibody according to any one of embodiments 1-8 and 24-29, an antigen-binding fragment according to any one of embodiments 9 and 24 to 30, a bispecific antibody according to any one of embodiments 31 to 33, or a T cell or NK cell according to any one of embodiments 36 and 37, to said subject.
42. A method for detecting the presence of metastatic cancer in a subject, the method comprising administering a labelled antibody or antigen-binding fragment thereof, which specifically recognizes nNaᵥ1.5, to the subject and subsequently measuring signal distribution derived from the labelled antibody or antigen-binding fragment in said subject, where a localised dense signal in a part of the body of the subject is indicative of the presence of metastatic disease in said part of the body.
43. A method for detecting the presence of neonatal 5′-exon splice variants of the α subunit of Nav1.5 (nNav1.5) in a biological sample from a subject, the method comprising contacting the biological sample with an antibody or antigen-binding fragment thereof which specifically recognizes nNaᵥ1.5, and subsequently detecting binding of the antibody or antigen-binding fragment to the biological sample.
44. The method according to any one of embodiments 39 to 42, wherein the subject is selected from the group consisting of
   a subject having a high risk of developing metastatic cancer, such as a patient having a familiar disposition for metastatic cancer and
   a cancer patient who has undergone surgical, medical, and/or radiation anti-cancer therapy without showing clinical signs of metastatic disease.
45. The method according to any one of embodiments 39 to 44, wherein the cancer is selected from solid tumours and non-solid tumours.
46. The method according to any one of embodiments 39 to 45, wherein the cancer is a cancer of a tissue selected from the lung, prostate, stomach, breast, large intestine, rectum, ovary, pancreas, liver, and CNS, such as the brain.
47. The method according to embodiment 46, wherein the cancer is cancer of the breast or colon or ovary.
48. The method according to any one of embodiments 39 to 47, wherein the cancer is colorectal cancer, breast cancer, lung cancer, prostate cancer, ovarian cancer, neuroblastoma or astrocytoma, or a combination of any thereof.
49. The monoclonal antibody according to any one of embodiments 1-8 and 24-30, the antigen binding fragment according to any one of embodiments 9 and 24-30, a bispecific antibody according to any one of embodiments 33 to 35, or a T cell or NK cell according to any one of embodiments 36 and 37 for use in a method according to any one embodiments 38 to 44.

The invention is further illustrated by the following Example, which should not be interpreted as limiting.

### EXAMPLES

### Example 1—Monoclonal Antibodies Against nNav1.5

Briefly, the peptide described in Chioni et al., (2005); NH₂-VSENIKLGNLSALRC-amide (SEQ ID NO:5), herein referred to as “NESO peptide”, was used as the immunizing peptide in laboratory mice. Hybridomas were obtained using standard hybridoma technology (NS0 cell line murine myeloma cells), and the following clones/subclones prepared (murine IgG1 kappa antibodies):
Clone 7/Subclone: 7-88
Clone 58/Subclone: 58-39
Clone A3/Subclone: A3-2
Clone C2/Subclone: C2-2

The mAb candidates from these clones/subclones were evaluated by peptide-based and cell-based ELISAs. The former incorporated the original immunizing peptide. The latter employed the human breast cancer MDA-MB-231 cells (Fraser et al., 2005).

Additionally, immunocytochemical tests of the mAbs were carried out using a pair of EBNA-293 cell lines stably expressing nNav1.5 and aNav1.5, described in Chioni et al. (2005). See FIG. 2 for the results.

Further details can be found below.

### 1. Immunization

1. Two Balb/c mice (M904 and M906) were immunized using the nNav1.5-specific peptide used earlier for the production of the polyclonal antibody, NESOpAb (Chioni et al., 2005). About 10 mg of peptide was synthesized with purity of >80%. Of this, 3-4 mg of peptide was conjugated to a carrier protein (ovalbumin, KLH or other).
2. Mice were immunized according to the protocol shown in Table 2.

TABLE 2
Immunization protocol

| DAY | Procedure |
|---|---|
| 1 | 100 μg carrier-conjugated peptide + |
| | Complete Freund's Adjuvant Intraperitoneal injection |
| 21 | 50 μg carrier-conjugated peptide + |
| | Incomplete Freund's Adjuvantn Intraperitoneal injection |
| 35 | 50 μg carrier-conjugated peptide + |
| | Incomplete Freund's Adjuvant Intraperitoneal injection |
| 49 | 50 μg carrier-conjugated peptide + |
| | Incomplete Freund's Adjuvant Intraperitoneal injection |
| 63 | 100 μg carrier-conjugated peptide + |
| | Incomplete Freund's Adjuvant Intravenous injection |

### 2. Cell Fusion

Three days after the final intravenous injection, mice were sacrificed by cervical dislocation. Prior to sacrificing, blood was collected from each mouse and serum was analyzed by ELISA, confirming positivity for the binding with the immobilized antigen.
1. Spleens were surgically resected. Cell fusion was performed, according to the standard method of Köhler and Milstein (1975). Spleens of both M904 and M906 were used.
2. To obtain spleen cell suspension, spleens were minced in a sterile Petri dish, using a surgical scalpel, then they were homogenized in 2 ml of DMEM culture medium (Euroclone) using a glass tissue homogenizer.
3. B-lymphocytes isolated from a spleen after the homogenization procedure were fused with aminopterine sensitive myeloma cells (NS0) using the polyethylene glycol (PEG) method (Galfrè and Milstein, 1981).
4. NS0 cells were maintained in logarithmic phase growth for 7 days before cell fusion. An equal number (108 cells) of B-lymphocytes and NS0 cells were mixed together in 30 ml of DMEM+4 mM L-Gln and centrifuged at 700 g for 5 min. Supernatant was discarded and the tube with cell pellet was placed in a beaker with 37 °C water.
5. The cell fusion protocol was as follows:
   Add 1 ml of 37 °C preheated PEG in 1 minutes, stirring;
   Stir for 2 minutes;
   Add 1 ml of 37 °C preheated DMEM+4 mM L-Gln in 1 minutes, stirring;
   Repeat above;
   Add 1 ml of 37 °C preheated DMEM+4 mM L-Gln in 30 sec, stirring;
   Repeat above;
   Add 6 ml of 37 °C preheated DMEM+4 mM L-Gln in 2 minutes, stirring;
   Add 12 ml of 37 °C preheated DMEM+4 mM L-Gln drop to drop, stirring;
   Centrifuge at 800 g for 5 minutes and discard supernatant.
6. The fusion product was resuspended in DMEM+4 mM L-Gln supplemented with 20% FetalClone I serum (Hyclone) and HAT, (Hypoxanthine, Aminopterine, Timidine) (Sigma).

B-cell/NS0 hybrid selection requires the use of a selective agent, added to the medium, which is HAT. HAT contributes to selection, exploiting the following mechanism: NS0 myeloma cells lack the expression of Hypoxanthine-Guanine PhosphoRibosyl Transferase (HGPRT) enzyme. HGPRT is an enzyme that catalyses the conversion of hypoxanthine to inosine monophosphate and guanine to guanosine monophosphate, transferring the 5-phosphoribosyl group from 5-phosphoribosyl 1-pyrophosphate (PRPP) to the purine, thus playing a central role in the generation of purine nucleotides through the purine salvage pathway. Cells that do not express this enzyme can produce purine nucleotides only through the de novo pathway; but the latter is blocked by aminopterine.
7. The cell fusion product was resuspended in 48 ml of selective complete DMEM medium (supplemented with the selective agent HAT) and aliquoted, according to the following dilutions 1:2, 1:4, 1:8 and 1:8 in 24-well multi-well plates (2 ml for well), labelled as follows:
   M904, dilution 1:2
   M904, dilution 1:4
   M904, dilution 1:8A
   M904, dilution 1:8B
   M906, dilution 1:2
   M906, dilution 1:4
   M906, dilution 1:8A
   M906, dilution 1:8B
8. The plates were incubated in a humidified incubator (37 °C, 5% CO₂). After two weeks, the selected hybridomas formed visible colony-like clusters.
9. Hybridoma colonies derived from 1:2 and 1:4 dilutions were grown. Supernatant (1 ml) was collected from each well and analyzed for their capacity to bind the antigen by Enzyme-Linked Immunosorbent Assay (ELISA) in order to select the best population for soft agar cloning.

### 3. Initial Screening

1. Hybridoma supernatants (×96) collected from dilutions 1:2 and 1:4 of both M904 and M906 were analyzed by peptide-based ELISA (enzyme linked immunosorbent assay) to assess the binding to the immobilized antigen.
2. ELISA assay was performed using nNav1.5-peptide, previously employed for the immunization, as antigen. The antigen was diluted to 10 μg/ml in 100 mM Na₂CO₃ buffer and aliquoted 100 μl/well in a 96-well plate. After incubation overnight at 4 °C, wells were quenched by addition of 200 μl/well blocking solution (3% BSA in 0.05% Tween-20 in PBS) and rested for one hour at room temperature. After further washing, each supernatant was dispensed in duplicate, 100 μl/well. After incubation for two hours at room temperature and washing, anti-mouse secondary antibody diluted in blocking solution 1:500 (10 μg/ml) was dispensed 100 μl/well and incubated for one hour at room temperature. The assay reaction was developed by addition of 100 μl/well TMB (3,3′,5,5′-Tetramethylbenzidine) substrate. The reaction was stopped with 100 μl/well 0.5 M HCl. Plates were analyzed measuring the absorbance at 450 nm.
3. 16 positive clones (OD450≥0.8) were obtained; of these, 7 clones were strongly positive (OD450≥1) (Table 3).

TABLE 3

| Serial No. | MOUSE | CLONE | DILUTION | OD450 |
|---|---|---|---|---|
| 1 | M904 | A1 | 1:2 | 0.881 |
| 2 | M904 | A2 | 1:2 | 0.865 |
| 3 | M904 | A3 | 1:2 | 1.250 |
| 4 | M904 | A4 | 1:2 | 2.006 |
| 5 | M904 | A6 | 1:2 | 0.892 |
| 6 | M904 | B1 | 1:2 | 0.845 |
| 7 | M904 | B6 | 1:2 | 0.868 |
| 8 | M904 | C1 | 1:2 | 0.858 |
| 9 | M904 | C2 | 1:2 | 1.967 |
| 10 | M904 | C5 | 1:2 | 1.830 |
| 11 | M904 | D1 | 1:2 | 1.151 |
| 12 | M904 | D2 | 1:2 | 0.879 |
| 13 | M904 | D5 | 1:2 | 1.853 |
| 14 | M904 | C2 | 1:4 | 0.836 |
| 15 | M904 | C5 | 1:4 | 0.950 |
| 16 | M904 | D4 | 1:4 | 1.636 |
| 17 | M906 | A3 | 1:8A | 1.603 |
| 18 | M906 | C2 | 1:8A | 1.075 |
| 19 | M906 | A6 | 1:8B | 1.422 |

4. A further dilution (1:8) was also tested on supernatants (divided into two plates, A and B). From these, 3 clones emerged as strongly positive (OD450≥1):
M906-A3-1:8A (1.603)
M906-C2-1:8A (1.075)
M906-A6-1:81B (1.422)
(Table 1)
5. All 19 positive clones were amplified in flasks. Supernatants were collected and frozen.

### 4. Cell-Based ELISA Screening on Positive and Negative Clones

1. Cell-based ELISAs were performed on MDA-MB-231 cells by the method of Sette et al. (2013). Cells were seeded and grown to semi-confluence in 96-well plates and incubated overnight in an incubator (37 °C, 5% CO₂).
2. After washing the cells three times with PBS, antibody supernatants were added to the cells and incubated for 2 hours at room temperature. The following steps were as for the peptide-based ELISAs. The results obtained from cell-based ELISA assay on MDA-MB 231 cells are shown in Table 4.

TABLE 4
The table summarizes the results obtained in
cell-based ELISA analyzing 10 clones
(5 positive and 5 negative from peptide-based ELISAs).

| Clone | OD450 |
|---|---|
| BLANK | −0.114 |
| M904-D5-1:2 | 0.274 |
| M904-C2-1:2 | 0.080 |
| M906-A6-1:8B | 0.134 |
| M906-A4-1:8° | 0.095 |
| M904-C1-1:2 | 0.133 |
| M904-C5-1:2 | 0.157 |
| M906-A3-1:8A | 0.676 |
| M906-C2-1:8A | 0.472 |
| M904-D4-1:4 | 0.506 |
| M906-D4-1:8B | 0.168 |

The clones below continued to give positive results both in peptide-based ELISA and cell-based ELISA assay.
M904-D5-1:2
M906-A3-1:8A
M906-C2-1:8A
M904-D4-1:4

### Notes:

1) M904-C2-1:2 gave poor absorbance signal in cell-ELISA (while it previously gave positive signal in peptide-based ELISA), probably due to weak confluence of cells in the well.
2) The three clones highlighted in bold, underlined text in Table 4 continuously gave positive results both in peptide-based ELISA and cell-based ELISA.

### 5. Further Cell-Based Screening

1. Of the 19 clones/supernatants tested on MDA-MB-231 cells, the following 5 gave a good fluorescence signal:
M904-A3-1:2
M904-D5-1:2
M904-C5-1:4
M904-D3-1:2
M906-C2-1:8A.
2. Immunocytochemistry (ICC) assay: Cells were fixed in 2% PFA for 5 min and blocked with 10% swine serumin PBS (pH 7.4) for 1 h. The supernatant (at a given dilution) was applied for 1 h in a moist chamber and washed (3×5 min with washing buffer containing 0.1% BSA in PBS; pH 7.4). The secondary anti-body (FITC-conjugated; diluted 1:50 in 5% swine serum in PBS; pH 7.4) was then applied for 1 h and then washed off, as described above. Finally, cells were washed in distilled H2O and mounted with anti-fading mounting medium (Vectashield, Vector, Burlingame, USA) and visualised with a Zeiss Axioscop-2 fluorescent micro-scope. As a positive control, NESOpAb was used. In negative controls, primary antibody (supernatant) was omitted. All these procedures were performed at room temperature (Chioni et al., 2005).
3. The ICC assay was repeated on MDA-MB-231 for the 5 positive clones and 3 negative clones. Then, the 8 clones were tested on SW620 cells (which also express nNav1.5) with the same technique. The positive clones that showed a good staining were:
M904-A3-1:2 and M906-C2-1:8A.
4. The results of the ICC on MDA-MB-231 and SW620 cells are summarized in Table 5. SW620 cells are human colon cancer cells, known to be invasive and to express nNav1.5. Clones that were positive for MDA-MB-231 cells also gave a good signal for SW620 cells.

TABLE 5

| | | | | | | Immuno- | |
|---|---|---|---|---|---|---|---|
| | | | | | | fluorescence | |
| | Immuno-fluorescence intensity | | | | | intensity | |
| | MDA-MB-231 | | | | | SW620 | |
| nNav 1.5 CLONE NAME | IICC | II ICC | III ICC | IV ICC | V ICC | I ICC | II ICC |
| MOUSE 904, A3, 1:2 | ++ | ++ | +++ | ++ | +++ | ++ | +++ |
| MOUSE 904, C1, 1:2 | − | − | − | − | − | − | − |
| MOUSE 904, D5, 1:2 | ++ | + | ++ | ++ | ++ | + | + |
| MOUSE 904, C2, 1:4 | − | − | − | − | − | − | − |
| MOUSE 906, C2, 1:4 | − | − | − | − | − | − | − |
| MOUSE 904, C5, 1:4 | ++ | ++ | +++ | ++ | | + | + |
| MOUSE 906, C2, 1:8A | ++ | ++ | +++ | + | | ++ | +++ |
| MOUSE 904 D3 1:2 | ++ | + | ++ | ++ | | + | + |

−: complete absence of signal
+: weak intensity
++: moderate intensity
+++: strong intensity

5. The 5 positive clones (for MDA-MB-231) were also tested on transfected EBNA-293 cells expressing neonatal or adult Nav1.5 channel isoform (Table 6).

TABLE 6

| | Immuso-fluorescence intensity | | | immuno-fluorescence intensity | | |
|---|---|---|---|---|---|---|
| | EBNA neonatal nNav1.5 | | | EBNA adult nNav1.5 | | |
| nNav 1.5 CLONE NAME | IICC | II ICC | III ICC | I ICC | II ICC | III ICC |
| MOUSE 904, A3, 1:2 | +++ | +++ | ++++ | − | + | − |
| MOUSE 904, C1, 12 | − | − | − | − | − | − |
| MOUSE 904, D5, 1:2 | + | + | + | +− | + | + |
| MOUSE 904, C2, 1:4 | − | − | − | − | − | − |
| MOUSE 906, C2, 1:4 | − | − | − | − | − | − |
| MOUSE 904, C5, 1:4 | +++ | + | + | − | + | + |
| MOUSE 906, C2, 1:8A | +++ | +++ | +++ | − | − | − |
| MOUSE 904 D3 1:2 | − | − | + | + | − | + |

−: complete absence of signal
+: weak intensity
+++: strong intensity

6. From the ICC analysis on EBNA cells, it emerged that the following two clones had the best selectivity for neonatal vs. adult Nav1.5:
M904-A3-1:2 and
M906-C2-1:8A

The fluorescence intensity signals for nNav1.5 for these two clones (12±2.5 and 17±2.6 a.u., respectively) were comparable to that obtained with NESOpAb (20±3.5).

These were then subject to PCR amplification and sequencing.

### 6. PCR Amplification

For the amplification of VH and VL regions (of the A3 clone), a 5′ primer that anneals to the VH and VL framework 1 (FR1) (primer forward) and a primer that anneals to the constant region adjacent to VH and VL domains (primer reverse) were chosen. For VL, a degenerate primer able to recognize the kappa light chain was designed. For VH, a 20-base degenerate primer that anneals to the IgG1 heavy chain was designed. The primers used are shown in Table 6 (described originally by Wang et al., 2000).

TABLE 6
Primer sequences for VH and VL

| Target | Primer sequence | SEQ ID NO |
|---|---|---|
| VH | | |
| Forward | CAGGTTACTCTGAAAGWGTSTG | 6 |
| (degH1dir) | | |
| Reverse | GGAAGATCTATAGACAGATGGGGGTGTCGTTTTGGC | 7 |
| (IgG1rev) | | |
| VL | | |
| Forward | GAYATTGTGMTSACMCARWCTMCA | 8 |
| (degKappadir) | | |
| Reverse | GGATACAGTTGGTGCAGCATC | 9 |
| (Kapparev) | | |

The amplification reaction of VH and VL was performed with the Phusion. High-25 Fidelity DNA Polymerase (Finnzymes Reagents), following the manufacturer's instructions. The PCR cycles were as shown in Table 7.

TABLE 7
PCR cycle used for the amplification of VH and VL mRNAs

| Step | Process | Temperature (° C.) | Time (Minutes) |
|---|---|---|---|
| 1 | Initial denaturation | 94 | 2 |
| 2 | Denaturation | 94 | 0.5 (30 seconds) |
| 3 | Annealing | 56 (VH) | 1 |
| | | 48 (VL) | 1 |
| 4 | Extension | 72 | 1 |
| 5 | Final extension | 72 | 10 |
| + | Steps 2-4 were | | |
| | repeated 24 times | | |

### 7. VH and VL Cloning

PCR products relative to VH and VL were run on 1% agarose in TAE buffer (Tris, acetic acid and EDTA), then excised from the gel with a scalpel and purified using QIAquick PCR Purification Kit (QIAGEN), according to the manufacturer's instruction. QIAquick Kits contain a silica membrane assembly for binding of DNA in high-salt buffer and elution with water. The purification procedure removes impurities from DNA samples. After purification from agarose gel, PCR products were cloned into pCR™—Blunt vector (where it is possible to insert blunt PCR products), using a 10:1 molar ratio of insert:vector, according to the following formula:

X (ng) insert=(10)(Y bp PCR product)(25 ng linearized pCR™−Blunt)/(3500 bp PCR™−Blunt),

where X ng is the amount of PCR product of Y base pairs to be ligated (Ausubel et al., 1994). The ligation reaction was incubated at 25 °C for 15 min. TOP10F cells were used for the transformation step and the selected colonies were checked for the presence of the insert in the right orientation.

### 8. Sequencing and Analysis of VH and VL

DNA samples were sequenced by Eurofins Genomics and the relative products were analyzed using IMGTTool software in order to find the three scFv Complementary Determining Regions (CDR1, CDR2 and CDR3). 12 colonies for VH and 12 colonies for VL were screened and the results showed that they are equal.

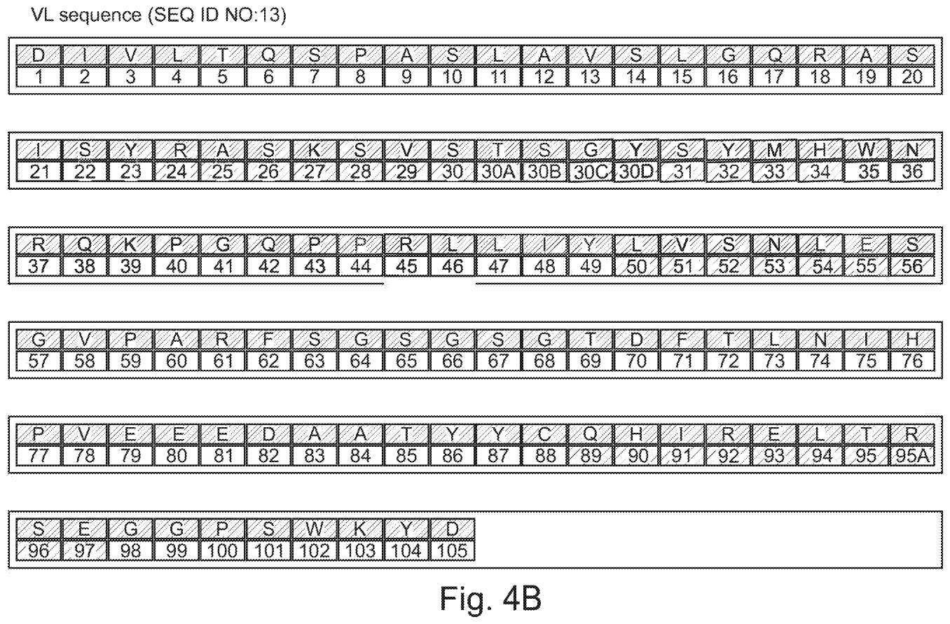

The VH and VL nucleotide sequences (SEQ ID NO:10 and 11, respectively) of the A3 antibody are shown in FIG. 4.

### REFERENCES FOR EXAMPLE 1

Ausubel, F. M., Brent, R., Kingston, R. E., Moore, D. D., Seidman, J. G., Smith, J. A., and Struhl, K. (1994). Current Protocols in Molecular Biology (New York: Greene Publishing Associates and WileyInterscience).
Chioni A M, Fraser S P, Pani F, Foran P, Wilkin G P, Diss J K, Djamgoz M B. A novel polyclonal antibody specific for the Na(v)1.5 voltage-gated Na(+) channel ‘neonatal’ splice form. J Neurosci Methods. 2005 Sep. 30; 147(2):88-98. doi: 10.1016/j.jneumeth.2005.03.010.
Galfrè G, Milstein C. Preparation of monoclonal antibodies: strategies and procedures. Methods Enzymol. 1981; 73(Pt B):3-46. doi: 10.1016/0076-6879(81)73054-4.
Köhler G, Milstein C. Continuous cultures of fused cells secreting antibody of predefined specificity. Nature. 1975 Aug. 7; 256(5517):495-7. doi: 10.1038/256495α0.
Sette A, Spadavecchia J, Landoulsi J, Casale S, Haye B, Crociani O, Arcangeli A. doi: 10.1007/s11051-013-2111-6.
Wang Z, Raifu M, Howard M, Smith L, Hansen D, Goldsby R et al. Universal PCR amplification of mouse immunoglobulin gene variable regions: the design of degenerate primers and an assessment of the effect of DNA polymerase 3′ to 5′ exonuclease activity, J Immunol Methods 2000; 233:167-77.

SEQUENCE TABLE

| SEQ ID NO/ | |
|---|---|
| Description | Sequence |
| SEQ ID NO: 1 | MANFLLPRGTSSFRRFTRESLAAIEKRMAEKQARGSTTLQESREGLPEEEAPRPQLDLQAS |
| aNav1.5 | KKLPDLYGNPPQELIGEPLEDLDPFYSTQKTFIVLNKGKTIFRFSATNALYVLSPFHPIRR |
| UniProtKB- | AAVKILVHSLFNMLIMCTILTNCVFMAQHDPPPWTKYVEYTFTAIYTFESLVKILARGFCL |
| Q14524 | HAFTFLRDPWNWLDFSVIIMAYTTEFVDLGNVSALRTFRVLRALKTISVISGLKTIVGALI |
| (SCN5A_HUMAN), | QSVKKLADVMVLTVFCLSVFALIGLQLFMGNLRHKCVRNFTALNGTNGSVEADGLVWESLD |
| ISOFORM-1 | LYLSDPENYLLKNGTSDVLLCGNSSDAGTCPEGYRCLKAGENPDHGYTSFDSFAWAFLALF |
| (Nav1.5c) | RLMTQDCWERLYQQTLRSAGKIYMIFFMLVIFLGSFYLVNLILAVVAMAYEEQNQATIAET |
| >sp\|Q14524\| | EEKEKRFQEAMEMLKKEHEALTIRGVDTVSRSSLEMSPLAPVNSHERRSKRRKRMSSGTEE |
| SCN5A_HUMAN | CGEDRLPKSDSEDGPRAMNHLSLTRGLSRTSMKPRSSRGSIFTFRRRDLGSEADFADDENS |
| Sodium channel | TAGESESHHTSLLVPWPLRRTSAQGQPSPGTSAPGHALHGKKNSTVDCNGVVSLLGAGDPE |
| protein type 5 | ATSPGSHLLRPVMLEHPPDTTTPSEEPGGPQMLTSQAPCVDGFEEPGARQRALSAVSVLTS |
| subunit alpha | ALEELEESRHKCPPCWNRLAQRYLIWECCPLWMSIKQGVKLVVMDPFTDLTITMCIVLNTL |
| OS = Homo | FMALEHYNMTSEFEEMLQVGNLVFTGIFTAEMTFKIIALDPYYYFQQGWNIFDSIIVILSL |
| sapiens | MELGLSRMSNLSVLRSFRLLRVFKLAKSWPTLNTLIKIIGNSVGALGNLTLVLAIIVFIFA |
| Ox = 9606 | VVGMQLFGKNYSELRDSDSGLLPRWHMMDFFHAFLIIFRILCGEWIETMWDCMEVSGQSLC |
| GN = SCN5A PE = 1 | LLVFLLVMVIGNLVVLNLFLALLLSSFSADNLTAPDEDREMNNLQLALARIQRGLRFVKRT |
| SV = 2 | TWDFCCGLLRQRPQKPAALAAQGQLPSCIATPYSPPPPETEKVPPTRKETRFEEGEQPGQG |
| | TPGDPEPVCVPIAVAESDTDDQEEDEENSLGTEEESSKQQESQPVSGGPEAPPDSRTWSQV |
| | SATASSEAEASASQADWRQQWKAEPQAPGCGETPEDSCSEGSTADMTNTAELLEQIPDLGQ |
| | DVKDPEDCFTEGCVRRCPCCAVDTTQAPGKVWWRLRKTCYHIVEHSWFETFIIFMILLSSG |
| | ALAFEDIYLEERKTIKVLLEYADKMFTYVFVLEMLLKWVAYGFKKYFTNAWCWLDFLIVDV |
| | SLVSLVANTLGFAEMGPIKSLRTLRALRPLRALSRFEGMRVVVNALVGAIPSIMNVLLVCL |
| | IFWLIFSIMGVNLFAGKFGRCINQTEGDLPLNYTIVNNKSQCESLNLTGELYWTKVKVNFD |
| | NVGAGYLALLQVATFKGWMDIMYAAVDSRGYEEQPQWEYNLYMYIYFVIFIIFGSFFTLNL |
| | FIGVIIDNFNQQKKKLGGQDIFMTEEQKKYYNAMKKLGSKKPQKPIPRPLNKYQGFIFDIV |
| | TKQAFDVTIMFLICLNMVTMMVETDDQSPEKINILAKINLLFVAIFTGECIVKLAALRHYY |
| | FTNSWNIFDFVVVILSIVGTVLSDIIQKYFFSPTLFRVIRLARIGRILRLIRGAKGIRTLL |
| | FALMMSLPALFNIGLLLFLVMFIYSIFGMANFAYVKWEAGIDDMFNFQTFANSMLCLFQIT |
| | TSAGWDGLLSPILNTGPPYCDPTLPNSNGSRGDCGSPAVGILFFTTYIIISFLIVVNMYIA |
| | IILENFSVATEESTEPLSEDDFDMFYEIWEKFDPEATQFIEYSVLSDFADALSEPLRIAKP |
| | NQISLINMDLPMVSGDRIHCMDILFAFTKRVLGESGEMDALKIQMEEKFMAANPSKISYEP |
| | ITTTLRRKHEEVSAMVIQRAFRRHLLQRSLKHASFLFRQQAGSGLSEEDAPEREGLIAYVM |
| | SENFSRPLGPPSSSSISSTSFPPSYDSVTRATSDNLQVRGSDYSHSEDLADFPPSPDRDRE |
| | SIV |
| SEQ ID NO: 2 | MANFLLPRGTSSFRRFTRESLAAIEKRMAEKQARGSTTLQESREGLPEEEAPRPQLDLQAS |
| nNav1.5 | KKLPDLYGNPPQELIGEPLEDLDPFYSTQKTFIVLNKGKTIFRFSATNALYVLSPFHPIRR |
| (example) | AAVKILVHSLFNMLIMCTILTNCVFMAQHDPPPWTKYVEYTFTAIYTFESLVKILARGFCL |
| UniProtKB- | HAFTFLRDPWNWLDFSVIIMAYVSENIKLGNLSALRTFRVLRALKTISVIPGLKTIVGALI |
| H9KVD2 | QSVKKLADVMVLTVFCLSVFALIGLQLFMGNLRHKCVRNFTALNGTNGSVEADGLVWESLD |
| (H9KVD2_HUMAN) | LYLSDPENYLLKNGTSDVLLCGNSSDAGTCPEGYRCLKAGENPDHGYTSFDSFAWAFLALF |
| >tr\|H9KVD2\| | RLMTQDCWERLYQQTLRSAGKIYMIFFMLVIFLGSFYLVNLILAVVAMAYEEQNQATIAET |
| H9KVD2_HUMAN | EEKEKRFQEAMEMLKKEHEALTIRGVDTVSRSSLEMSPLAPVNSHERRSKRRKRMSSGTEE |
| Sodium channel | CGEDRLPKSDSEDGPRAMNHLSLTRGLSRTSMKPRSSRGSIFTFRRRDLGSEADFADDENS |
| protein | TAGESESHHTSLLVPWPLRRTSAQGQPSPGTSAPGHALHGKKNSTVDCNGVVSLLGAGDPE |
| OS = Homo | ATSPGSHLLRPVMLEHPPDTTTPSEEPGGPQMLTSQAPCVDGFEEPGARQRALSAVSVLTS |
| sapiens | ALEELEESRHKCPPCWNRLAQRYLIWECCPLWMSIKQGVKLVVMDPFTDLTITMCIVLNTL |
| Ox = 9606 | FMALEHYNMTSEFEEMLQVGNLVFTGIFTAEMTFKIIALDPYYYFQQGWNIFDSIIVILSL |
| GN = SCN5A PE = 1 | MELGLSRMSNLSVLRSFRLLRVFKLAKSWPTLNTLIKIIGNSVGALGNLTLVLAIIVFIFA |
| SV = 1 | VVGMQLFGKNYSELRDSDSGLLPRWHMMDFFHAFLIIFRILCGEWIETMWDCMEVSGQSLC |
| | LLVFLLVMVIGNLVVLNLFLALLLSSFSADNLTAPDEDREMNNLQLALARIQRGLRFVKRT |
| | TWDFCCGLLRQRPQKPAALAAQGQLPSCIATPYSPPPPETEKVPPTRKETRFEEGEQPGQG |
| | TPGDPEPVCVPIAVAESDTDDQEEDEENSLGTEEESSKQQESQPVSGGPEAPPDSRTWSQV |
| | SATASSEAEASASQADWRQQWKAEPQAPGCGETPEDSCSEGSTADMTNTAELLEQIPDLGQ |
| | DVKDPEDCFTEGCVRRCPCCAVDTTQAPGKVWWRLRKTCYHIVEHSWFETFIIFMILLSSG |
| | ALAFEDIYLEERKTIKVLLEYADKMFTYVFVLEMLLKWVAYGFKKYFTNAWCWLDFLIVDV |
| | SLVSLVANTLGFAEMGPIKSLRTLRALRPLRALSRFEGMRVVVNALVGAIPSIMNVLLVCL |
| | IFWLIFSIMGVNLFAGKFGRCINQTEGDLPLNYTIVNNKSQCESLNLTGELYWTKVKVNFD |
| | NVGAGYLALLQVATFKGWMDIMYAAVDSRGYEEQPQWEYNLYMYIYFVIFIIFGSFFTLNL |
| | FIGVIIDNFNQQKKKLGGQDIFMTEEQKKYYNAMKKLGSKKPQKPIPRPLNKYQGFIFDIV |
| | TKQAFDVTIMFLICLNMVTMMVETDDQSPEKINILAKINLLFVAIFTGECIVKLAALRHYY |
| | FTNSWNIFDFVVVILSIVGTVLSDIIQKYFFSPTLFRVIRLARIGRILRLIRGAKGIRTLL |
| | FALMMSLPALFNIGLLLFLVMFIYSIFGMANFAYVKWEAGIDDMFNFQTFANSMLCLFQIT |
| | TSAGWDGLLSPILNTGPPYCDPTLPNSNGSRGDCGSPAVGILFFTTYIIISFLIVVNMYIA |
| | IILENFSVATEESTEPLSEDDFDMFYEIWEKFDPEATQFIEYSVLSDFADALSEPLRIAKP |
| | NQISLINMDLPMVSGDRIHCMDILFAFTKRVLGESGEMDALKIQMEEKFMAANPSKISYEP |
| | ITTTLRRKHEEVSAMVIQRAFRRHLLQRSLKHASFLFRQQAGSGLSEEDAPEREGLIAYVM |
| | SENFSRPLGPPSSSSISSTSFPPSYDSVTRATSDNLQVRGSDYSHSEDLADFPPSPDRDRE |
| | SIV |
| SEQ ID NO: 3/ | YVSENIKLGNLSALRTFRVLRALKTISVIP |
| Amino acid | |
| sequence | |
| corresponding to | |
| exon 6 of a | |
| nNav1.5 | |
| SEQ ID NO: 4/ | YVTEFVXLGNVSALRTFRVLRALKTISVIP, wherein ″X″ is K or D |
| Partial consensus | |
| sequence | |
| nNav1.5 and | |
| aNav1.5 (FIG. 1) | |
| SEQ ID NO: 5/ | NH₂-VSENIKLGNLSALRC-amide |
| NESO peptide | |
| SEQ ID NO: 6 | CAGGTTACTCTGAAAGWGTSTG |
| VH primer; | |
| Forward | |
| (degH1dir) | |
| SEQ ID NO: 7 | GGAAGATCTATAGACAGATGGGGGTGTCGTTTTGGC |
| VH primer; | |
| Reverse | |
| (IgG1rev) | |
| SEQ ID NO: 8 | GAYATTGTGMTSACMCARWCTMCA |
| VL primer; | |
| Forward | |
| (degKappadir) | |
| SEQ ID NO: 9 | GGATACAGTTGGTGCAGCATC |
| VL primer; | |
| Reverse | |
| (Kapparev) | |
| SEQ ID NO: 10 | CAGGTTACTCTGAAAGTGTCTGGCCCTGGGATATTGCAGCCCTCCCAGACCCTCAGTCTGA |
| A3 VH sequence | CCTGTTCTTTTTCTGGGTTTTCACTGACCACTTCTGGTATGGGTGTGAGCTGGATTCGTCA |
| (nucleotide) | GGCTGCAGGAAAGGGTCTGGAGTGGCTGGCACACATTTACTGGGATGATGACAAGCGCTAT |
| | AACCCAGCCCTGAAGAGCCGGCTCACAATCTCCAAGGATACCTCCAGCAACCAAGTATTCC |
| | TCAACATCACCAGTGTGGACACTGCAGATACTGCCACATACTACTGTGCTCGAAGGGAGGA |
| | CTATGTTACGAGTTTGCTAAGTGGGGCCAAGGGACTCTGGTCACTGTGTTTG |
| SEQ ID NO: 11 | GATATTGTGCTGACCCAGTCTCCTGCTTCCTTAGCTGTATCTCTGGGGCAGAGGGCCAGCA |
| A3 VL sequence | TCTCATACAGGGCCAGCAAAAGTGTCAGTACATCTGGCTATAGTTATATGCACTGGAACCG |
| (nucleotide) | ACAGAAGCCAGGACAGCCACCCAGACTCCTCATCTATCTTGTATCCAACCTAGAATCTGGG |
| | GTCCCTGCCAGGTTCAGTGGCAGTGGGTCTGGGACAGACTTCACCCTCAACATCCATCCTG |
| | TGGAGGAGGAGGATGCTGCAACCTATTACTGTCAGCACATTAGGGAGCTTACACGTTCGGA |
| | GGGGGGACCAAGCTGGAAATACGAC |
| SEQ ID NO: 12 | QVTLKVSGPGILQPSQTLSLTCSFSGFSLTTSGMGVSWIRQAAGKGLEWLAHIYWDDDKRY |
| A3 VH sequence | NPALKSRLTISKDTSSNQVFLNITSVDTADTATYYCARREDYVTSLLSGAKGLWSLCL |
| (protein) | |
| SEQ ID NO: 13 | DIVLTQSPASLAVSLGQRASISYRASKSVSTSGYSYMHWNRQKPGQPPRLLIYLVSNLESG |
| A3 VL sequence | VPARFSGSGSGTDFTLNIHPVEEEDAATYYCQHIRELTRSEGGPSWKYD |
| (protein) | |
| SEQ ID NO: 14 | GFSLTTS |
| VH CDR1 | |
| SEQ ID NO: 15 | YWDDD |
| VH CDR2 | |
| SEQ ID NO: 16 | REDYVTSLL |
| VH CDR3 | |
| SEQ ID NO: 17 | RASKSVSTSGYSYMH |
| VL CDR1 | |
| SEQ ID NO: 18 | LVSNLES |
| VL CDR2 | |
| SEQ ID NO: 19 | QHIRELTRSE |
| VL CDR3 | |

### REFERENCES

Each reference cited in the present disclosure or listed below is incorporated by reference, in its entirety.
Brackenbury et al., Cancer Res Treat. 2007 January; 101(2):149-60. doi: 10.1007/s10549-006-9281-1.
Dotti et al., Immunol Rev. 2014 January; 257(1): doi:10.1111/imr.12131
Sadelain et al., Cancer Discov. 2013 April; 3(4):388-398. doi:10.1158/2159-8290.CD-12-0548.
Chioni et al., J Neuroscience Methods 2005; 147:88-98.
Fraser et al., Clin Cancer Res 2005; 11:5381-5389.
Fraser et al., Pflugers Archives 2003; 446:559-71.
Grimes et al., FEBS Lett 1995; 369:290-294.
Laniado et al., The Prostate 2001; 46:262-274.
Laniado et al., Am J Pathol 1997; 150:1213-1221.
Onkal et al., J Cell Physiol 2008; 216:716-726.
Brisson et al., J Cell Sci. 2013 Nov. 1; 126(Pt 21):4835-42. doi: 10.1242/jcs.123901. Epub 2013 Jul. 31.
Djamgoz et al., Cancers (Basel). 2019 Oct. 28; 11(11). pii: E1675. doi: 10.3390/cancers11111675.

1. A method of treatment or amelioration of metastatic cancer in a subject comprising the steps of
a) administering a labelled antibody or antigen-binding fragment thereof which specifically recognizes neonatal Nav1.5 (nNav1.5),
b) detecting the in vivo signal distribution derived from the labelled nNav1.5 specific antibody or antigen-binding fragment in a tissue of the subject, and
c) administering a nNav1.5 voltage-gated sodium channel (VGSC) current inhibitor at a therapeutically effective dosage to the subject when the measured signal distribution exhibits a localized signal.

2. A method for detecting the presence of neonatal 5′-exon splice variants of the α subunit of Nav1.5 (nNav1.5) in a biological sample from a subject, the method comprising contacting the biological sample with an antibody or antigen-binding fragment thereof which specifically recognizes nNav1.5, and subsequently detecting binding of the antibody or antigen-binding fragment to the biological sample.

3. The method according to claim 2, wherein the sample is a biological sample being a cancer cell-containing tumor biopsy or blood sample.

4. A method for treatment or amelioration of metastatic cancer in a subject, comprising administering an effective amount of an antibody or antigen-binding fragment thereof, which specifically recognizes the neonatal 5′-exon splice variants of the α subunit of Nav1.5 (nNav1.5).

5. The method according to claim 1, wherein the antibody or antigen-binding fragment binds specifically to neonatal 5′-exon splice variants of the α subunit of Naᵥ1.5 (nNaᵥ1.5).

6. The method according to claim 5, wherein the antibody or antigen-binding fragment binds to the peptide segment corresponding to residues 206 to 219 of SEQ ID NO:2.

7. The method according to claim 6, wherein the nNaᵥ1.5 is a human nNaᵥ1.5.

8. The method according to claim 7, wherein the antibody or antigen-binding fragment binds more readily to a nNaᵥ1.5 comprising the amino acid sequence of residues 206 to 219 of SEQ ID NO:2 than to an adult 3-exon splice variant of the α subunit of Naᵥ1.5 (aNaᵥ1.5) comprising the amino acid sequence of residues 206 to 219 of SEQ ID NO:1.

9. The method according to claim 8, wherein the antibody or antigen-binding fragment comprises a VH region at least 80%, 90%, 95%, 97%, 98% or 99% identical, to the VH region encoded by SEQ ID NO:10, and a VL region at least 80%, 90%, 95%, 97%, 98% or 99% identical, to the VL region encoded by SEQ ID NO:11.

10. The method according to claim 9, wherein the antibody or antigen-binding fragment comprises a VH region comprising complementarity determining region (CDR) 1, CDR2, and CDR3 amino acid sequences comprising SEQ ID NOs: 14, 15, and 16, respectively.

11. The method according to claim 10, wherein the antibody or antigen-binding fragment comprises a VL region comprising CDR1, CDR2, and CDR3 amino acid sequences comprising SEQ ID NOs: 17, 18, and 19, respectively.

12. The method according to claim 11, wherein the antibody or antigen-binding fragment is chimeric or humanized.

13. The method according to claim 12, wherein all constant domains, framework regions, or constant domains and framework regions of the antibody or antigen-binding fragment are human.

14. The method according to claim 1, wherein the cancer is a cancer of a tissue selected from the lung, prostate, stomach, breast, large intestine, rectum, ovary, pancreas, liver, and CNS, such as the brain.

15. The method according to claim 14, wherein the cancer is cancer of the breast or colon or ovary.

16. The method according to claim 14, wherein the cancer is colorectal cancer, breast cancer, lung cancer, prostate cancer, ovarian cancer, neuroblastoma or astrocytoma, or a combination of any thereof.

17. The method according to claim 14, wherein the cancer expresses nNav1.5 or is a cancer associated with a risk for nNav1.5-expression.

18. The method according to claim 1, wherein the subject is selected from the group consisting of:
a) a subject having a high risk of developing metastatic cancer, such as a patient having a familial disposition for metastatic cancer; and
b) a cancer patient who has undergone surgical, medical, and/or radiation anti-cancer therapy without showing clinical signs of metastatic disease.

19. The method according to claim 1, wherein the nNav1.5 VGSC current inhibitor is selected from ranolazine, eleclazine and an antibody or antigen-binding fragment binds specifically to neonatal 5′-exon splice variants of the α subunit of Naᵥ1.5 (nNaᵥ1.5).

20. The method according to claim 19, wherein the nNav1.5 VGSC current inhibitor is a humanized monoclonal antibody specific for the neonatal 5′-exon splice variants of the α subunit of Naᵥ1.5 (nNaᵥ1.5), the antibody comprising a VH region comprising complementarity determining region (CDR) 1, CDR2, and CDR3 amino acid sequences comprising SEQ ID NOs: 14, 15, and 16, respectively and a VL region comprising CDR1, CDR2, and CDR3 amino acid sequences comprising SEQ ID NOs: 17, 18, and 19, respectively.


SEQUENCE LISTING (19 sequences)

SEQ ID NO: 1 - protein, 2016 aa, Homo sapiens [sequence omitted]

SEQ ID NO: 2 - protein, 2016 aa, Homo sapiens
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPIRRAAVKILVHSLFNMLIMCTILTNCVFMAQHDPPPWTKYVEYTFTAIYTFESLVKILARGFCLHAFTFLRDPWNWLDFSVIIMAYVSENIKLGNLSALRTFRVLRALKTISVIPGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTAGESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRQRPQKPAALAAQGQLPSCIATPYSPPPPETEKVPPTRKETRFEEGEQPGQGTPGDPEPVCVPIAVAESDTDDQEEDEENSLGTEEESSKQQESQPVSGGPEAPPDSRTWSQVSATASSEAEASASQADWRQQWKAEPQAPGCGETPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVATFKGWMDIMYAAVDSRGYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 3 - protein, 30 aa, Homo sapiens
YVSENIKLGNLSALRTFRVLRALKTISVIP

SEQ ID NO: 4 - protein, 30 aa, synthetic construct
  REGION 1..30: note = Consensus sequence of Homo sapiens variants
  VARIANT 7: note = X is K or D
YVTEFVXLGNVSALRTFRVLRALKTISVIP

SEQ ID NO: 5 - protein, 15 aa, synthetic construct
  REGION 1..15: note = NESO peptide sequence
  MOD_RES 15: note = AMIDATION
VSENIKLGNLSALRC

SEQ ID NO: 6 - DNA, 22 bp, synthetic construct
  misc_feature 1..22: note = Primer sequence
  source 1..22: mol_type = other DNA
caggttactctgaaagwgtstg

SEQ ID NO: 7 - DNA, 36 bp, synthetic construct
  misc_feature 1..36: note = Primer sequence
  source 1..36: mol_type = other DNA
ggaagatctatagacagatgggggtgtcgttttggc

SEQ ID NO: 8 - DNA, 24 bp, synthetic construct
  misc_feature 1..24: note = Primer sequence
  source 1..24: mol_type = other DNA
gayattgtgmtsacmcarwctmca

SEQ ID NO: 9 - DNA, 21 bp, synthetic construct
  misc_feature 1..21: note = Primer sequence
  source 1..21: mol_type = other DNA
ggatacagttggtgcagcatc

SEQ ID NO: 10 - DNA, 357 bp, Mus musculus
  source 1..357: mol_type = other DNA
caggttactctgaaagtgtctggccctgggatattgcagccctcccagaccctcagtctgacctgttctttttctgggttttcactgaccacttctggtatgggtgtgagctggattcgtcaggctgcaggaaagggtctggagtggctggcacacatttactgggatgatgacaagcgctataacccagccctgaagagccggctcacaatctccaaggatacctccagcaaccaagtattcctcaacatcaccagtgtggacactgcagatactgccacatactactgtgctcgaagggaggactatgttacgagtttgctaagtggggccaagggactctggtcactgtgtttg

SEQ ID NO: 11 - DNA, 330 bp, Mus musculus
  source 1..330: mol_type = other DNA
gatattgtgctgacccagtctcctgcttccttagctgtatctctggggcagagggccagcatctcatacagggccagcaaaagtgtcagtacatctggctatagttatatgcactggaaccgacagaagccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatacgac

SEQ ID NO: 12 - protein, 119 aa, Mus musculus
QVTLKVSGPGILQPSQTLSLTCSFSGFSLTTSGMGVSWIRQAAGKGLEWLAHIYWDDDKRYNPALKSRLTISKDTSSNQVFLNITSVDTADTATYYCARREDYVTSLLSGAKGLWSLCL

SEQ ID NO: 13 - protein, 110 aa, Mus musculus
DIVLTQSPASLAVSLGQRASISYRASKSVSTSGYSYMHWNRQKPGQPPRLLIYLVSNLESGVPARFSGSGSGTDFTLNIHPVEEEDAATYYCQHIRELTRSEGGPSWKYD

SEQ ID NO: 14 - protein, 7 aa, Mus musculus
GFSLTTS

SEQ ID NO: 15 - protein, 5 aa, Mus musculus
YWDDD

SEQ ID NO: 16 - protein, 9 aa, Mus musculus
REDYVTSLL

SEQ ID NO: 17 - protein, 15 aa, Mus musculus
RASKSVSTSGYSYMH

SEQ ID NO: 18 - protein, 7 aa, Mus musculus
LVSNLES

SEQ ID NO: 19 - protein, 10 aa, Mus musculus
QHIRELTRSE